(19) United States
(12) Patent Application Publication
(10) Pub. No.: US 2026/0098306 A1
(43) Pub. Date: Apr. 9, 2026

(54) METHOD AND KIT TO DETERMINE TISSUE OR CELL ORIGIN OF CFDNA AND USE THEREOF TO TRACE TISSUE DAMAGE IN DISEASES

(71) Applicants: THE UNIVERSITY OF HONG KONG, Hong Kong (CN); Centre for Oncology and Immunology Limited, Hong Kong SAR (CN)

(72) Inventors: Philip Hei LI, Ma On Shan (CN); Chak Sing LAU, Pokfulam (CN); Guang Sheng LING, Pokfulam (CN); Wing Hon Jason WONG, Pokfulam (CN); Qiuyu JING, Hong Kong (CN)

(73) Assignees: THE UNIVERSITY OF HONG KONG, Hong Kong (CN); Centre for Oncology and Immunology Limited, Hong Kong SAR (CN)

(21) Appl. No.: 19/350,905

(22) Filed: Oct. 6, 2025

Related U.S. Application Data
(60) Provisional application No. 63/704,370, filed on Oct. 7, 2024.

(51) Int. Cl. C12Q 1/6886; C12Q 1/6883

(52) U.S. Cl. CPC C12Q 1/6886; C12Q 1/6883; C12Q 2600/154

(57) ABSTRACT

Provided is a method to determine the tissue or cell origin of cfDNA from a sample obtained from a subject and uses thereof to trace tissue damage in disease and disorder in the subject. The method comprises measuring read-level or fragment-level cfDNA methylation at cell type or tissue-specific regions and assigning cfDNA to a cell type or tissue origin. The cell type or tissue specific cfDNA level indicates cell or tissue damage in the subject. Also provided is the use of the present method for developing targeted kits for diagnosis, patient screening,

### CROSS-REFERENCE TO RELATED PATENT APPLICATIONS

This application claims priority to U.S. Provisional Application Ser. No. 63/704,370 filed on Oct. 7, 2024, which is incorporated by reference herein in its entirety.

### 1. FIELD

Provided is a method to determine the tissue or cell origin of cfDNA from a sample obtained from a subject and uses thereof to trace tissue damage in disease and disorder in the subject.

### 2. BACKGROUND

Cell-free DNA (cfDNA) are DNA fragments circulating in blood, urine and other body fluids. It's thought that these cfDNA molecules are released from dying cells in the body. Tracing the tissue or cell type origin of cfDNA is thus important and can be used to indicate specific organ or tissue involvement in physiological and pathological conditions. Benefiting from next-generation sequencing, it's able to detect fetal cfDNA in maternal blood and therefore allow for non-invasive prenatal test (NIPT) ¹⁻². Donor-derived cfDNA can be used to identify graft rejection in organ-transplant patients³⁻⁵. Moreover, cfDNA is an important biomarker for screening and diagnosis of multiple cancers⁶⁻⁷.

Currently, several methods can be used to trace tissue origin of cfDNA. One is based on the endogenous and exogenous differences, such as chromosome abnormality and single nucleotide polymorphisms (SNPs) used for NIPT and graft rejection prediction. The other popular method is based on DNA methylation signals. DNA methylation, especially the methylation at cytosine adjacent to guanine (CpG) sites, is important for regulation of cell type specific gene expression and is thus a fundamental tissue/cell type marker⁸⁻¹⁰. Relative contribution of tissues or cell types to cfDNA can be calculated by fitting cfDNA methylation profiles against a matrix of marker methylation panel¹⁰⁻¹¹. This can be achieved by non-negative least square (NNLS) models or other models¹²⁻¹³. All of the models rely on the methylation matrix panels profiled either by whole-genome bisulfite sequencing (WGBS) or targeted methylation profiling of marker regions. As such, the relative abundance of one tissue or cell type is always affected by the methylation level at other cell type marker regions.

The present inventors discovered new methods to deconvolute the contribution of different tissues and cell types to a DNA mixture based on methylation levels at specific genomic sites. The accuracy and sensitivity of the methods is demonstrated with synthesized mixture data. Furthermore, this method can be used to trace the damage of specific tissues and enable the tissue/cell type-derived DNA components as promising biomarker for diseases.

In one aspect, the method in the present disclosure identify tissue-specific or cell-specific damage associated with diseases by tracing the tissue or cell origin of circulating cell-free DNA (cfDNA). In one embodiment, kidney injury in patients with systemic lupus erythematosus (SLE) is detected. In one embodiment, the method utilizes a kidney-specific DNA methylation signature to deconvolute cfDNA and infer kidney-derived contributions. In one embodiment, kidney methylation signature was derived from comparisons of methylation profiles across healthy cell types and therefore represents tissue identity rather than a methylation change caused by SLE. In short, the kidney methylation pattern used in the method is not itself a disease-associated marker for SLE.

In one aspect, the present invention relates to a method of determining if a subject has suffered tissue damage from a disease or disorder. In certain embodiments, the method comprises: (a) measuring read-level or fragment-level cfDNA methylation in a sample from the subject; (b) normalizing and assigning the cell type or tissue-specific origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the subject has suffered or suffers tissue damage from the exposure.

The present disclosure provides methods to profile the abundance or contribution of specific tissue or cell type contribution to a DNA mixture, such as cfDNA.

The present disclosure provides applications of the described methods to detect tissue or cell type involvement in physiological and pathological conditions, including but not limited to autoimmune diseases and cancers.

The present disclosure provides targeted methylation profiling kits derived from the described methods or the idea involved in the described methods to capture specific tissue or cell type contributions to a DNA mixture, such as cfDNA. The kits can be based on targeted methylation sequencing, probe capturing, methylation array and beyond.

Also provided is a method of treating a subject, comprising: (a) receiving a plurality of sequencing reads for a cell-free deoxyribonucleic acid (cfDNA) sample obtained or derived from the subject, wherein each of the plurality of sequencing reads comprises methylation sequencing data obtained from a nucleic acid sequence; (b) determining a methylation pattern for a sequencing read in the plurality of sequencing reads, wherein the methylation pattern comprises a genomic region corresponding to the nucleic acid sequence and methylation status of one or more motifs in the genomic region; (c) measuring the quantity of the cfDNA sample as containing cfDNAs derived from a tissue from the subject with a disorder, based on reads ratio or RPKM, thereby identifying the subject as having the disorder indicated by the tissue; and (d) administering a treatment to the subject based on the identifying the subject as having the disorder.

In certain embodiments, the normal quantity of cfDNA comprises a quantity of cfDNA for the determined cellular origin that is generated in a population of individuals who do not have a disease or disorder.

In one aspect, the present disclosure relates to a method of treating tissue damage in a subject.

In one aspect, provided is a method of treating a subject having a cell, tissue or organ damage from a disease or disorder, the method comprising: (a) obtaining a sample from the subject containing cfDNA; (b) receiving a plurality of read-level or fragment-level cell-free deoxyribonucleic acid (“cfDNA”) methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; (e) determining the reads ratio or RPKM as compared to a control sample from a subject without the cell or tissue damage, wherein the reads ratio or RPKM above a threshold range indicates that the subject has damage in the specific cell-type or tissue; and (f) administering a treatment to the subject based on the identifying the subject as having the disorder.

In certain embodiments, the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

In certain embodiments, the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

In certain embodiments, wherein the diseases or disorder indicates a cell, organ and tissue damage from graft rejections in organ transplantation, immune related diseases, lupus nephritis, myocarditis, infection, cancer and radiation induced damages.

In certain embodiments, the cfDNA sample is obtained or derived from a plasma sample, a blood sample, a saliva sample, an amniotic fluid sample, a cystic fluid sample, a spinal fluid sample, a brain fluid sample, a urine sample, a sweat sample, or a tears sample from the subject.

In certain embodiments, the organ or tissue is adipose, bladder, colon, skin, stomach, heart, kidney, liver, lung, brain, pancreas, prostate, small intestine, thyroid or a combination thereof.

In certain embodiments, the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

In certain embodiments, the treatment is chemotherapy, radiation therapy, immunotherapy, target therapy and tumor resection.

In certain embodiments, the diseases or disorder indicates a cell, organ and tissue damage from graft rejections in organ transplantation, immune related diseases, lupus nephritis, myocarditis, infection, cancer and radiation induced damages.

In one aspect, provided is a method of identifying tissue-specific damage in a subject having a disease or disorder, comprising: (a) receiving a plurality of sequencing reads for a cell-free deoxyribonucleic acid (cfDNA) sample obtained or derived from the subject, wherein each of the plurality of sequencing reads comprises methylation sequencing data obtained from a nucleic acid sequence; (b) determining a methylation pattern for a sequencing read in the plurality of sequencing reads, wherein the methylation pattern comprises a genomic region corresponding to the nucleic acid sequence and methylation status of one or more motifs in the genomic region; (c) characterizing the cfDNA sample as containing cfDNAs derived from a tissue of the subject based on a reads ratio or RPKM, wherein the characterization of the cfDNA as being derived from the tissue of the subject indicates tissue-specific damage to the tissue of the subject.

In one aspect, provided is a method of monitoring a subject having a cell or tissue or organ damage from a disease or disorder after treatment, wherein the monitoring comprises at least two times, the method comprising: (a) obtaining a sample from the subject containing cfDNA; (b) receiving a plurality of read-level or fragment-level cell-free deoxyribonucleic acid (“cfDNA”) methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; (e) determining the reads ratio or RPKM as compared to a control sample from a subject without the cell or tissue damage, wherein the reads ratio or RPKM having an above threshold range prior to a first treatment indicates that the subject has damage in the specific cell-type or tissue, and wherein the reads ratio or RPKM after a first time point after a first treatment having a below threshold range indicates the treatment is effective, and wherein the reads ratio or RPKM after a second time point after the first treatment having a higher threshold range indicates that the treatment is effective at the first time point but recurred at the second time point.

In certain embodiments, the first and second time point is 5 days, 10 days, 2 weeks, 3 weeks, 4 weeks, 5 weeks, 6 weeks, 3 months, 6 months, 1 year, 2 years, 3 years, 4 years, 5 years, 6 years, 10 years, 15 years, 20 years or more.

In certain embodiments, the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

In certain embodiments, the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

In certain embodiments, the disorder could be varieties of organ and tissue involved pathological conditions, including but not limited to graft rejections in organ transplantations, immune related diseases such as lupus nephritis and myocarditis, infection, cancer and radiation induced tissue damages of the subject.

In certain embodiments, the cfDNA sample is obtained or derived from body fluids, including but not limited to a plasma sample, a blood sample, a saliva sample, an amniotic fluid sample, a cystic fluid sample, a spinal fluid sample, a brain fluid sample, a urine sample, a sweat sample, or a tears sample from the subject.

In certain embodiments, the organ or tissue is adipose, bladder, colon, skin, stomach, heart, kidney, liver, lung, brain, pancreas, prostate, small intestine, thyroid or a combination thereof.

In certain embodiments, the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

In certain embodiments, the treatment is chemotherapy, radiation therapy, immunotherapy, target therapy and tumor resection.

### 4. BRIEF DESCRIPTION OF THE FIGURES

The patent or application file contain at least one drawing executed in color.

FIGS. 1A-C. Deconvolution of DNA components for simulated adipose-monocytes data A). Abundance of adipose. B). Abundance of monocytes. C). Within-group coefficient of variation of adipose abundance.

FIGS. 2A-C. Deconvolution of DNA components for simulated heart-monocytes data A). Abundance of heart. B). Abundance of monocytes. C). Within-group coefficient of variation of heart abundance.

FIGS. 3A-C. Deconvolution of DNA components for simulated kidney-monocytes data A). Abundance of kidney. B). Abundance of monocytes. C) Within-group coefficient of variation of kidney abundance.

FIGS. 4A-C. Deconvolution of DNA components for simulated liver-monocytes data A). Abundance of liver. B). Abundance of monocytes. C) Within-group coefficient of variation of liver abundance.

FIGS. 5A-C. Deconvolution of DNA components for simulated lung-monocytes data A). Abundance of lung. B). Abundance of monocytes. C) Within-group coefficient of variation of lung abundance.

FIGS. 6A-C. Deconvolution of DNA components for simulated pancreas-monocytes data A). Abundance of pancreas. B). Abundance of monocytes. C). Within-group coefficient of variation of pancreas abundance.

FIG. 7. Correlation of kidney cfDNA abundance between NNLS and custom methods.

FIG. 8. Increase of kidney cfDNA in active LN patients.

FIGS. 9A-C. Correlation of blood kidney cfDNA with A). SLEDAI score. B). C3 complement. C). C4 complement.

FIG. 10. Reference regions extracted from public data²¹ (Loyfer. N, et al. Nature, 2023). The arrow shows the regions for kidney as an example used for lupus nephritis detection among SLE patients.

FIG. 11. Workflow of the present method for cfDNA as biomarker for disease in accordance with one or more embodiments.

FIG. 12. Calculation of tissue/cell type composition of cfDNA sample.

### 4.1 Definitions

The term “source” refers to an origin of cfDNA. Sources may be human sources including human organ, tissue or cell types.

The term “cell free DNA,” or “cfDNA” refers to deoxyribonucleic acid fragments that circulate in an individual's body (e.g., blood).

The term “genomic nucleic acid,” “genomic DNA,” or “gDNA” refers to nucleic acid molecules or deoxyribonucleic acid molecules obtained from one or more cells.

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human (or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g. of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g. thyroid, breast), intraocular fluids (e.g. the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 gx10 minutes, obtaining the fluid part, and re-centrifuging at for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

As used herein, the terms “cell-free DNA” or “cfDNA” or “circulating cell-free DNA” refers to DNA that is circulating in the peripheral blood of a subject. The DNA molecules in cfDNA may have a median size that is no greater than 1 kb (for example, about 50 bp to 500 bp, or about 80 bp to 400 bp, or about 100 bp to 1 kb), although fragments having a median size outside of this range may be present. This term is intended to encompass free DNA molecules that are circulating in the bloodstream as well as DNA molecules that are present in extra-cellular vesicles (such as exosomes) that are circulating in the bloodstream.

A “sequence read” refers to a string of nucleotides sequenced from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed. An amount of sequence reads can be used as a proxy for the number of DNA fragments. To determine the number of DNA fragments from the amount of sequence reads, a calculation may be performed to account for paired-end sequencing and/or bias of sequencing techniques.

A “site” (also called a “genomic site”) corresponds to a single site, which may be a single base position or a group of correlated base positions, e.g., a CpG site, TSS site, Dnase hypersensitivity site, or larger group of correlated base positions. A “locus” may correspond to a region that includes multiple sites. A locus can include just one site, which would make the locus equivalent to a site in that context.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

Terms such as “treating” or “treatment” or “to treat” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

### 5. DETAILED DESCRIPTION

The present disclosure involves analysis of cfDNA to determine its cellular origin. Determination of the cellular origin of cfDNA comprises identifying methylation patterns in the sequence of the cfDNA and comparing the methylation patterns in the sequence of the cfDNA to know methylation patterns associated with different cell types.

Provided are methods to profile the tissue or cell type components of cfDNA and other DNA mixture, where the specific components are independent of other tissue or cell type marker regions. For example, in one or more embodiments the present methods can be applied to trace the kidney cfDNA in systematic lupus erythematosus (SLE) patients for the characterization of active lupus nephritis (LN). The present methods can also be applied to other disease states or disorders and used for developing targeted kits for diagnosis, patient screening, long-term monitoring as well as outcome assessment for diseases and disorders.

More specifically, provided are methods to detect tissue and cell type involvement in pathological and physiological conditions based on blood cell-free DNA methylation signals. By profiling the methylation in specific regions, this method is accurate to trace the tissue and cell type origin of blood cell-free DNA, especially sensitive to detect cfDNA components with low abundance. The methods have demonstrated promising performance in detecting kidney injury in systemic lupus erythematosus patients and the profiled kidney cfDNA can be a promising biomarker for monitoring lupus nephritis. The methods can be utilized to measure tissue and cell type involvement in other conditions, including but not limited to autoimmune diseases and cancers.

The methods provide an easy and affordable way to trace cell type and tissue origin of DNA mixture, such as blood cell-free DNA, and thus can be further used to profile cell type and tissue involvement in different diseases.

To measure the abundance of specific tissue or cell type in a DNA mixture, the methods directly profile the abundance of unmethylated reads in the form of relative fraction or reads per kilobases per million (RPKM) in corresponding marker regions. In contrast to non-negative least squares and many other models, which rely on a methylation matrix of marker regions for all reference cell types, the present methods only focus on targeted tissue or cell type marker regions. This makes the present methods more robust and can be less affected by methylation signals from other regions. As exemplified herein, the accuracy and significance of the present methods are demonstrated using whole-genome bisulfite sequencing (WGBS) data. Theoretically, the DNA methylation signals can be extracted from WGBS, EM-seq, targeted methylation sequencing or targeted methylation array data. By focusing on these cell type-specific regions with a total length of around 3 million bases, the cost for either bisulfite sequencing or methylation array will be much cheaper than whole genome methylation sequencing.

This method can accurately profile the abundance of cfDNA originating from a specific cell type by leveraging DNA methylation signals from exactly the cell-type marker regions without influence from other cell-type marker regions. This method is easier to apply and more affordable clinically.

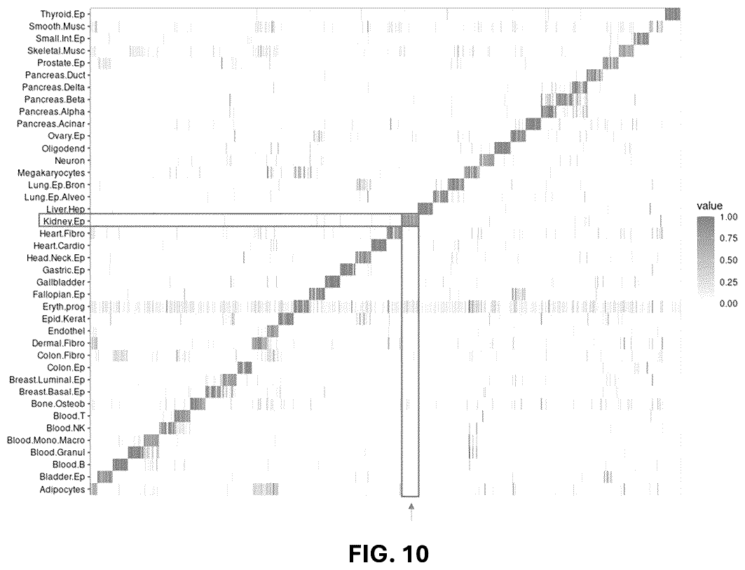

In order to determine the biological composition of cfDNA, a method (US 2023/0167507 A1) compares the cfDNA methylation pattern to pre-established methylation signature (the reference matrix), which comprises pre-determined signature region and methylation rate of that region. In contrast, the present disclosure determines the cfDNA composition by simply profiling the methylation pattern of cfDNA itself, with no need to compare the cfDNA methylation pattern to pre-determined signature. Although pre-determined signature regions which are extracted from published literature can be utilized in certain embodiments, these regions are used as reference in the present disclosure to determine in which region to profile and calculate the cfDNA methylation pattern (FIG. 10). There is no comparison between the cfDNA methylation pattern and the pre-established methylation signature in the methods of the present disclosure.

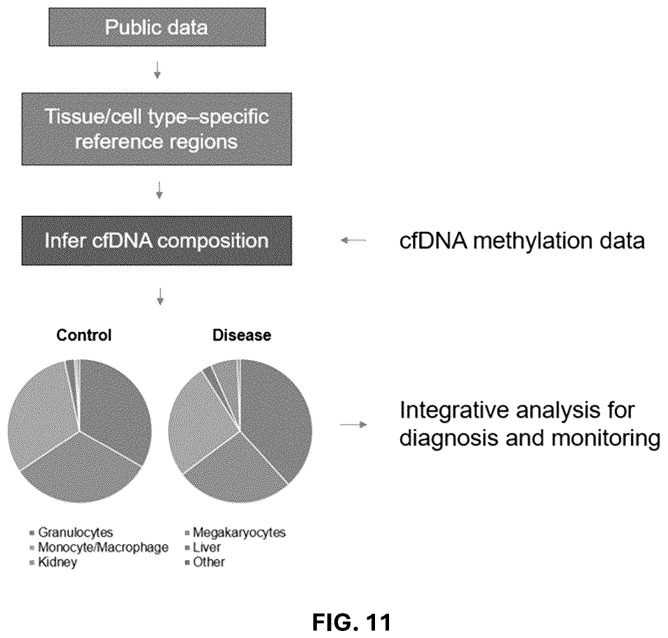

Reference regions were extracted from public datasets and profiled cfDNA methylation for the deconvolution of cfDNA composition. To evaluate the potential of cfDNA as biomarker for certain diseases, the cfDNA composition will be compared across diseased situation and controls (including healthy controls and possible other conditional controls). The basic workflow of the method in the present disclosure is shown in FIG. 11.

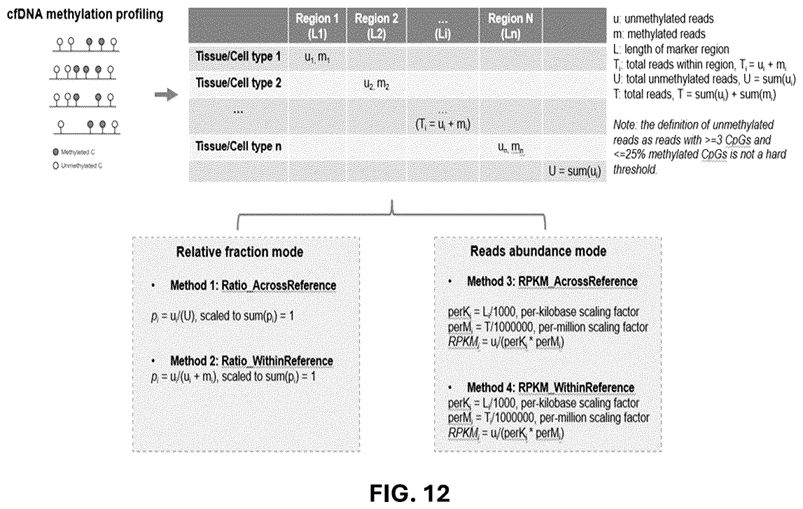

FIG. 12 illustrates one of the embodiments to calculate the tissue or cell type composition based on cfDNA methylation profiling. Since the tissue or cell type specific genomic regions are known from published literature, the cfDNA methylation level in these regions can be calculated based on the sequencing reads (the input cfDNA methylation profiling). Four methods have been provided to perform the calculation with different scaling and normalization strategies.

In one embodiment, disclosed is a method to determine tissue or cell origin of cfDNA in a sample from a subject. In one embodiment, the method is used to trace tissue damage in a subject having a disease or disorder.

In certain embodiments, provided herein is a workflow of applying the method for measuring tissue damage in a subject.

In certain embodiments, the method comprises the steps of: (i) providing circulating cell-free DNA (cfDNA) from a subject; (ii) measuring read-level or fragment-level cfDNA methylation at cell type or tissue-specific regions by comparing the read-level or fragment-level cfDNA methylation at cell type of tissue-specific regions in a control; (iii) assigning a cell type or tissue of origin based on read-level or fragment-level cfDNA methylation; (iv) measuring the quantity of cell type or tissue-specific cfDNA level, wherein if the quantity is higher than a threshold as compared to a control indicate cell or tissue damage in the subject.

The increase in the measured quantity of the cfDNA of the determined cellular origin over the normal quantity of cfDNA of the determined cellular origin, or over a previously measured quantity of cfDNA of the determined cellular origin, may be, for example, a percent increase of about 0.1% to 100%, such as about 0.1%, 0.5%, 1%, 2%, 3%, 4%, 5%, 6% 7%, 8%, 9%, 10%, 20%, 30%, 40%, 50%, 60%, 70%, 80%, 90%, or 100%; or may be a fold increase of at least about 2-fold, such as about 2-fold, or 3-fold, or 4-fold, or 5-fold, or 6-fold, or 7-fold, or 8-fold, or 9-fold, or 10-fold. In some embodiments, the increase may be any increase that is determined to be statistically significant (e.g., p≤005, p≤0.01, etc.) as calculated by statistical methods known in the art.

Methods for quantifying the cfDNA are known in the art and include, but are not limited to, PCR; fluorescence-based quantification methods (e.g., Qubit); chromatography techniques such as gas chromatography, supercritical fluid chromatography, and liquid chromatography, such as partition chromatography, adsorption chromatography, ion exchange chromatography, size exclusion chromatography, thin-layer chromatography, and affinity chromatography; electrophoresis techniques, such as capillary electrophoresis, capillary zone electrophoresis, capillary isoelectric focusing, capillary electrochromatography, micellar electrokinetic capillary chromatography, isotachophoresis, transient isotachophoresis, and capillary gel electrophoresis; comparative genomic hybridization; microarrays; and bead arrays.

Disclosed herein is the application of the method in detecting kidney damage associated with SLE and lupus nephritis.

In certain embodiments, the method is applied to identify other tissue or organ damage, such as lung damage in COVID-19 patients, liver damage in organ transplantation patients with allograft rejection and heart damage in autoimmune myocarditis.

In certain embodiments, varieties of cell, organ and tissue damages indicate disease or disorder, including but not limited to graft rejections in organ transplantation, immune related diseases such as lupus nephritis and myocarditis, infection, cancer and radiation induced damages.

In certain embodiments, the cell, organ or tissue damage indicates an exposure to a compound. In certain embodiments, the compound is a toxin.

In certain embodiments, the use of patterns of differential methylation to determine the cellular origin of cfDNA is applied to methods of treating a subject having a disease or disorder.

In other embodiments, the methods are for treating tissue damage in a subject. The methods comprise administering a treatment for tissue damage to the subject and monitoring the efficacy of the treatment.

In certain embodiments, the methods for treating tissue damage comprise administering a treatment for tissue damage to the subject and monitoring the efficacy of the treatment. The monitoring comprises, at two or more time points. A decrease in the measured quantity of the cfDNA of the determined cellular origin at a later time point as compared to an earlier time point is indicative that the treatment is effective. An increase or no change in the measured quantity of the cfDNA of the determined cellular origin at a later time point as compared to an earlier time point is indicative that the treatment is not effective.

In certain embodiments, the disease or disorder is lupus nephritis, active lupus nephritis, or systemic lupus erythematosus (“SLE”).

In certain embodiments, the organ damage is in the kidney.

In certain embodiments, the reads ratio across reference is ≥0.020.

In certain embodiments, the reads ratio within reference is ≥0.015.

In certain embodiments, the RPKM across reference is ≥12.

In certain embodiments, the RPKM across reference is ≥400.

In one or more embodiments, a method of monitoring a subject having a cell or tissue or organ damage from a disease or disorder after treatment is also provided. In certain embodiments, the monitoring comprises the following method, at least two times. Specifically, in certain embodiments, the method comprises: (a) obtaining a sample from the subject containing cfDNA; (b) receiving a plurality of read-level or fragment-level cfDNA methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; and (e) determining the reads ratio or RPKM as compared to a control sample from a subject without the cell or tissue damage. In certain embodiments, the reads ratio or RPKM having an above threshold range prior to a first treatment indicates that the subject has damage in the specific cell-type or tissue, and wherein the reads ratio or RPKM after a first time point after a first treatment having a below threshold range indicates the treatment is effective, and wherein the reads ratio or RPKM after a second time point after the first treatment having a higher threshold range indicates that the treatment is effective at the first time point but recurred at the second time point.

In certain embodiments, the amount of time between the first and second time point is 5 days, 10 days, 2 weeks, 3 weeks, 4 weeks, 5 weeks, 6 weeks, 3 months, 6 months, 1 year, 2 years, 3 years, 4 years, 5 years, 6 years, 10 years, 15 years, 20 years or more.

In certain embodiments, the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

In certain embodiments, the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

In at least one embodiment, the disorder could be varieties of organ and tissue involved pathological conditions, including but not limited to graft rejections in organ transplantations, immune related diseases such as lupus nephritis and myocarditis, infection, cancer and radiation induced tissue damages of the subject.

In certain embodiments, the cfDNA sample is obtained or derived from body fluids, including but not limited to a plasma sample, a blood sample, a saliva sample, an amniotic fluid sample, a cystic fluid sample, a spinal fluid sample, a brain fluid sample, a urine sample, a sweat sample, or a tears sample from the subject.

In certain embodiments, the organ or tissue is adipose, bladder, colon, skin, stomach, heart, kidney, liver, lung, brain, pancreas, prostate, small intestine, thyroid or a combination thereof.

In certain embodiments, the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

In certain embodiments, the treatment is chemotherapy, radiation therapy, immunotherapy, target therapy and tumor resection.

In certain embodiments, the disease or disorder is lupus nephritis, active lupus nephritis, or SLE. In certain embodiments, the organ damage is in the kidney.

Exemplary implementations of one or more steps of the above methods are provided below in the below examples.

### 6. EXAMPLES

### 6.1 Materials and Methods

### 6.1.1 Patient Recruitment and Data Collection

Public WGBS data of blood cfDNA from SLE patients¹⁴ was collected, where the SLE patients were recruited from Prince of Wales Hospital in Hong Kong with written informed consent. 10 SLE patients were also recruited from Queen Mary Hospital in Hong Kong. For each patient, 10 mL of peripheral blood were collected and separated plasma by centrifuge. 3 mL of plasma samples were sent to Novogene for cfDNA extraction and whole genome methylation profiling. The published dataset was combined with our own dataset for downstream analysis. In total, there are 30 samples collected from 30 SLE patients, 10 of them with active nephritis, 7 with LN remission and 13 never developing nephritis. 10 samples were also collected from 10 healthy donors as control. The patient recruitment and sample collection protocol was approved by the Institutional Review Board of the University of Hong Kong/Hospital Authority Hong Kong West Cluster (HKU/HA HKW IRB). This study was conducted in compliance with Declaration of Helsinki.

### 6.1.2 Simulation of DNA Mixture

WGBS data of adipose, kidney, liver, lung, monocytes and pancreas was downloaded from NCBI's Sequence Read Archive (SRA) (www.ncbi.nlm.nih.gov/sra). The detailed data source was listed in Table 1. Since monocytes is one of the major sources of blood cfDNA, adipose, kidney, liver, lung and pancreas WGBS data were randomly mixed with monocytes to reach a total of 100M of raw reads. In each mixture dataset, the proportion of target tissues are 0.5%, 1%, 2%, 5%, 10% and 20% respectively, with corresponding monocyte proportion as 99.5%, 99%, 98%, 95%, 90% and 80% respectively. Each combination was randomly repeated 10 times to count variability.

TABLE 1
Source of public WGBS data for DNA mixture simulation

| Tissue | Access No. |
|---|---|
| Adipose | SRR577617 |
| | SRR577618 |
| | SRR577619 |
| | SRR1045689 |
| | SRR1045690 |
| | SRR1045691 |
| Heart | SRR536242 |
| | SRR536243 |
| | SRR1045642 |
| | SRR1045643 |
| Kidney | SRR530648 |
| Liver | SRR641603 |
| | SRR641604 |
| | SRR641605 |
| Lung | SRR536237 |
| | SRR547636 |
| Monocytes | SRR1104855 |
| | SRR1104848 |
| | SRR1104857 |
| Pancreas | SRR547639 |
| | SRR1045706 |
| | SRR1045707 |
| | SRR1045708 |

### 6.1.3 Raw Data Processing

Adapters and low-quality reads were trimmed using Trim Galore (github.com/FelixKrueger/TrimGalore). Only reads with quality higher than 20 and length longer than 25 bp were kept. Bismark was applied to map reads to human genome hg19 and further for deduplication¹⁵.

### 6.1.4 Non-Negative Least Square (NNLS) Method for Deconvolution

wgbs_tools described by Loyfer, et al. 10 were implied, which applied conventional non-negative least square (NNLS) method for the deconvolution of cell type components of DNA mixture data. As described in the original paper, this method firstly defined unmethylated reads (U reads) as reads with less than or equal to 25% methylated CpGs out of at least 4 CpGs in total. The paper also constructed reference atlas A with 1,232 regions (top 25 markers per cell type), in which Aᵢⱼ cell holds the U proportion of ith marker in the jth cell type. For a given sample input, this method firstly calculated the proportion of U reads at each marker to form a 1,232×1 vector b. Then, it applied NNLS to infer coefficient vector x by minimizing |A x x-b|₂ subject to non-negative x and with sum of xⱼ to 1. The coefficient xⱼ is a representative of the relative contribution of jth cell type to the mixture of DNA. To test the performance of wgbstools, both the top 25 and top 250 markers for each cell type were tried.

### 6.1.5 Custom Methods for Deconvolution

Read-level methylation at cell type-specific marker regions was calculated using RLM¹⁶. Only reads with >=3 CpGs were kept for further analysis. Unmethylated reads were defined with <=25% methylated CpGs. Based on the specific hypomethylated marker regions identified by Loyfer, et al¹⁰, applied four different methods were applied, which are all independent of the reference methylation matrix A, to profile the cell type origins.

### 6.2 Workflow of Applying the Method to Measure Tissue Damage

In order to determine the tissue or cell origin of cfDNA, the following steps were implemented:
Step 1: provide circulating cell-free DNA (cfDNA).
Step 2: measure read- or fragment-level cfDNA methylation at cell type or tissue-specific regions.
Step 3: assign cfDNA read or fragment to a cell type or tissue of origin based on its read-level or fragment-level methylation.
Step 4: determine cell type or tissue-specific cfDNA level as a reflection of potential cell or tissue damage.

### 6.2.1 Experimental Protocol

Step 1: Provide cfDNA

CfDNA can be extracted from blood stream, urine or other body fluids using commercial kits, such as Qiagen's QIAamp Circulating Nucleic Acid Kit, Plasma/Serum Cell-Free Circulating DNA Purification Kits and so on, as well as other custom methods. The typical cfDNA input is 1-50 ng, or sometimes could be as low as undetectable. No upper limit restriction for cfDNA input.

Step 2: Measure Read- or Fragment-Level cfDNA Methylation at Cell Type or Tissue-Specific Regions

Both targeted and whole genome methylation profiling strategy can be applied to achieve this goal by applying either short-read sequencing or long-read sequencing. Fragment level methylation can be directly measured on long-read sequencing platform. While on short-read sequencing platform, both whole genome bisulfite sequencing (WGBS) and enzymatic methyl-seq (EM-seq) can be applied to measure whole genome methylation. In more details, read- or fragment-level cfDNA methylation can be profiled by any of the following methods or methods with similar strategies:

### 1. Short-Read WGBS for Whole Genome Methylation Profiling

cfDNA collected will go through bisulfite treatment to convert unmethylated cytosine to uracil. Converted DNA fragments will then be constructed into sequencing library using commercial kits or custom methods. Then the sequencing library will be sequenced on short-read sequencing platform, such as Illumina or BGI's sequencers, as well as other short-read sequencers to get at least 30 million non-redundant reads. The unmethylated cytosine (converted to uracil) will be read as thymine. Then the reads will be mapped to a reference genome. By comparing the C-to-T conversion compared to genome reference, methylation status can be identified at the original cytosine position. Only reads with >=3CpGs will be kept for further analysis. Each cytosine within the CpG locus will be classified as methylated (base-called as cytosine while it's also cytosine on the corresponding reference genome) or unmethylated (base-called as thymine while it's cytosine on the corresponding reference genome). At read level, unmethylated reads will be defined if there are <=25% methylated CpGs (reads will be labeled as u as indicated in FIG. 12). Otherwise, methylated reads will be labeled as m as indicated in FIG. 12.

### 2. Short-Read EM-Seq for Whole Genome Methylation Profiling

Provided cfDNA will go through enzyme treatment to convert unmethylated cytosine to uracil. Converted DNA fragments will then be constructed into sequencing library using commercial kits or custom methods. Then the sequencing library will be sequenced on short-read sequencing platform, such as Illumina or BGI's sequencers, as well as other short-read sequencers to get at least 30 million non-redundant reads. The unmethylated cytosine (converted to uracil) will be read as thymine. Then the reads will be mapped to a reference genome to determine the location of the reads and the bases. By comparing the C-to-T conversion compared to genome reference, methylation status can be identified at the original cytosine position. Only reads with >=3CpGs will be kept for further analysis. Each cytosine within the CpG locus will be classified as methylated (base-called as cytosine while it's also cytosine on the corresponding reference genome) or unmethylated (base-called as thymine while it's cytosine on the corresponding reference genome). At read level, unmethylated reads will be defined if there are <=25% methylated CpGs (reads will be labeled as u as indicated in FIG. 12). Otherwise, methylated reads will be labeled as m as indicated in FIG. 12.

### 3. Long-Read Sequencing Based on PacBio SMRT for Whole Genome Methylation Profiling

Provided cfDNA will be subject to hairpin adapter ligation to form circular DNA templates so called SMRTbell. The SMRTbell library can be loaded onto SMRT cells for sequencing. DNA methylation can be sequenced in real-time based on polymerase kinetics and can be called directly from the high-accuracy long reads (HiFi reads). Reads will also be mapped to reference genome to determine the genome location of the reads and bases. Only reads with >=3CpGs will be kept for further analysis. Each cytosine within the CpG locus will be classified as methylated or unmethylated directly from the raw signal. At read level, unmethylated reads will be defined if there are <=25% methylated CpGs (reads will be labeled as u as indicated in FIG. 12). Otherwise, methylated reads will be labeled as m as indicated in FIG. 12.

### 4. Long-Read Sequencing Based on Oxford Nanopore for Whole Genome Methylation Profiling

Provided cfDNA will be subject to adapter ligation to prepare sequencing library. The library can be loaded onto Nanopore flow cell for sequencing. DNA methylation can be sequenced in real-time based on electric signals. Methylation information for each cytosine on the reads can be called directly from the raw signal data. Reads will also be mapped to reference genome to determine the genome location of the reads and bases. Only reads with >=3CpGs will be kept for further analysis. Each cytosine within the CpG locus will be classified as methylated or unmethylated directly from the raw signal. At read level, unmethylated reads will be defined if there are <=25% methylated CpGs (reads will be labeled as u as indicated in FIG. 12). Otherwise, methylated reads will be labeled as m as indicated in FIG. 12.

### 5. Targeted Sequencing Based on Panel of Probes to Capture Specific to Cell Type or Tissue-Specific Marker Regions

To profile the methylation information for regions specific to cell type or tissue, panels of probes can be determined to capture cfDNA fragments with sequences specific to cell type or tissue marker regions. For example, we have designed a panel consisting of approximately 2000 probes targeting kidney-specific marker regions, covering around 250 regions with a total length of ˜64 kb (Target regions information shown in Table 2). The panel can be applied especially for short-read WGBS and short-read EM-seq.

For short-read WGBS and short-read EM-seq, after library construction as indicated above, the panel can be applied to incubate with the library for target capturing. After enrichment, the eluted library with go through another round of PCR amplification to get enough materials for sequencing on short-read sequencing platform as indicated above. The sequencing depth can be as low as 3 million non-redundant reads, which is around 10% of whole genome methylation profiling. Similar as that in whole genome methylation profiling, the unmethylated cytosine (converted to uracil) will be read as thymine. Then the reads will be mapped to a reference genome to determine the location of the reads and the bases. By comparing the C-to-T conversion compared to genome reference, methylation status can be identified at the original cytosine position. Only reads with >=3CpGs will be kept for further analysis. Each cytosine within the CpG locus will be classified as methylated (base-called as cytosine while it's also cytosine on the corresponding reference genome) or unmethylated (base-called as thymine while it's cytosine on the corresponding reference genome). At read level, unmethylated reads will be defined if there are <=25% methylated CpGs (reads will be labeled as u as indicated in FIG. 12). Otherwise, methylated reads will be labeled as m as indicated in FIG. 12.

The panel can also be applied to PacBio or Nanopore long-read sequencing library. After library construction as indicated above, the panel can be applied to incubate with the library for target capturing. After enrichment, the eluted library will be loaded on the corresponding long-read sequencer. DNA methylation can be sequenced in real-time. Similar as that in long-read whole genome methylation profiling, methylation information for each cytosine on the reads can be called directly from the raw signal data. Reads will also be mapped to reference genome to determine the genome location of the reads and bases. Only reads with >=3CpGs will be kept for further analysis. Each cytosine within the CpG locus will be classified as methylated or unmethylated directly from the raw signal. At read level, unmethylated reads will be defined if there are <=25% methylated CpGs (reads will be labeled as u as indicated in FIG. 12). Otherwise, methylated reads will be labeled as m as indicated in FIG. 12.

TABLE 2
Targeted region information

| chromosome | start | end | annotations |
|---|---|---|---|
| chr1 | 117715692 | 117715875 | gene_id VTCN1; transcript_id NM_001253849; |
| | | | gene_name VTCN1; |
| chr1 | 147058238 | 147058759 | gene_id BCL9; transcript_id NM_004326; gene_name |
| | | | BCL9; |
| chr1 | 151185300 | 151185528 | gene_id PIP5K1A; transcript_id NM_001135638; |
| | | | gene_name PIP5K1A; |
| chr1 | 171325352 | 171326008 | NA |
| chr1 | 175374907 | 175375330 | gene_id TNR; transcript_id NM_003285; gene_name |
| | | | TNR; |
| chr1 | 187041620 | 187041696 | NA |
| chr1 | 187041783 | 187041862 | NA |
| chr1 | 46131210 | 46131670 | gene_id GPBP1L1; transcript_id NM_021639; |
| | | | gene_name GPBP1L1; |
| chr1 | 5902819 | 5902963 | NA |
| chr1 | 6750284 | 6750650 | gene_id DNAJC11; transcript_id NM_018198; |
| | | | gene_name DNAJC11; |
| chr1 | 68075404 | 68075993 | NA |
| chr1 | 70661377 | 70661571 | gene_id LRRC40; transcript_id NM_017768; |
| | | | gene_name LRRC40; |
| chr10 | 117857749 | 117857831 | gene_id GFRA1; transcript_id NM_005264; gene_name |
| | | | GFRA1; |
| chr10 | 119543627 | 119543966 | NA |
| chr10 | 125969101 | 125969320 | NA |
| chr10 | 132891205 | 132891551 | gene_id TCERG1L; transcript_id NM_174937; |
| | | | exon_number 1; exon_id NM_174937.1; gene_name |
| | | | TCERG1L; |
| chr10 | 135120066 | 135120641 | gene_id TUBGCP2; transcript_id NR_046330; |
| | | | gene_name TUBGCP2; |
| chr10 | 13763955 | 13764295 | gene_id FRMD4A; transcript_id NM_001318337; |
| | | | gene_name FRMD4A; |
| chr10 | 1506477 | 1506548 | gene_id ADARB2; transcript_id NM_018702; |
| | | | gene_name ADARB2; |
| chr10 | 1514522 | 1514697 | gene_id ADARB2; transcript_id NM_018702; |
| | | | gene_name ADARB2; |
| chr10 | 1708667 | 1708788 | gene_id ADARB2; transcript_id NM_018702; |
| | | | gene_name ADARB2; |
| chr10 | 3248207 | 3248497 | NA |
| chr10 | 58680436 | 58680544 | NA |
| chr10 | 80538472 | 80538790 | NA |
| chr10 | 972671 | 972945 | gene_id LARP4B; transcript_id NM_015155; |
| | | | gene_name LARP4B; |
| chr10 | 99251294 | 99251664 | gene_id MMS19; transcript_id NM_001351359; |
| | | | gene_name MMS19; |
| chr11 | 11203899 | 11204023 | NA |
| chr11 | 11626479 | 11626712 | gene_id GALNT18; transcript_id NM_198516; |
| | | | gene_name GALNT18; |
| chr11 | 132031595 | 132031715 | gene_id NTM; transcript_id NM_001144058; |
| | | | gene_name NTM; |
| chr11 | 13246977 | 13247200 | NA |
| chr11 | 19371970 | 19372015 | NA |
| chr11 | 19645700 | 19645789 | gene_id NAV2; transcript_id NM_001111018; |
| | | | gene_name NAV2; |
| chr11 | 27970403 | 27970759 | NA |
| chr11 | 35515942 | 35516177 | gene_id PAMR1; transcript_id NM_001282676; |
| | | | gene_name PAMR1; |
| chr11 | 64733146 | 64733345 | gene_id MAJIN; transcript_id NM_001300803; |
| | | | gene_name MAJIN; |
| chr11 | 70855156 | 70855468 | gene_id SHANK2; transcript_id NM_012309; |
| | | | gene_name SHANK2; |
| chr11 | 71435951 | 71436148 | NA |
| chr11 | 939169 | 939468 | gene_id AP2A2; transcript_id NR_144510; gene_name |
| | | | AP2A2; |
| chr11 | 945369 | 945585 | gene_id AP2A2; transcript_id NR_144510; gene_name |
| | | | AP2A2; |
| chr12 | 116682622 | 116683192 | gene_id MED13L; transcript_id NM_015335; |
| | | | gene_name MED13L; |
| chr12 | 120403441 | 120403545 | NA |
| chr12 | 121036218 | 121036385 | NA |
| chr12 | 124388479 | 124388930 | gene_id DNAH10; transcript_id NM_207437; |
| | | | gene_name DNAH10; |
| chr12 | 127052610 | 127052780 | NA |
| chr12 | 127756296 | 127756380 | NA |
| chr12 | 131507099 | 131507260 | gene_id ADGRD1; transcript_id NM_001330497; |
| | | | gene_name ADGRD1; |
| chr12 | 131572350 | 131572700 | gene_id ADGRD1; transcript_id NM_001330497; |
| | | | gene_name ADGRD1; |
| chr12 | 4554786 | 4554972 | NA |
| chr12 | 57211212 | 57211488 | NA |
| chr12 | 7121904 | 7122192 | gene_id LPCAT3; transcript_id NM_005768; |
| | | | gene_name LPCAT3; |
| chr13 | 113314224 | 113314355 | gene_id ATP11AUN; transcript_id NR_164109; |
| | | | gene_name ATP11AUN; |
| chr13 | 21085810 | 21086060 | gene_id CRYL1; transcript_id NM_015974; gene_name |
| | | | CRYL1; |
| chr13 | 23422688 | 23422758 | NA |
| chr13 | 23422787 | 23422840 | NA |
| chr13 | 23422878 | 23422953 | NA |
| chr13 | 23423085 | 23423309 | NA |
| chr13 | 25026065 | 25026411 | gene_id PARP4; transcript_id NM_006437; gene_name |
| | | | PARP4; |
| chr13 | 33764942 | 33765270 | gene_id STARD13; transcript_id NM_001243474; |
| | | | gene_name STARD13; |
| chr13 | 49397756 | 49397983 | NA |
| chr13 | 76871766 | 76871871 | NA |
| chr13 | 79508572 | 79508633 | NA |
| chr13 | 96085429 | 96085970 | gene_id CLDN10; transcript_id NM_001160100; |
| | | | exon_number 1; exon_id NM_001160100.1; gene_name |
| | | | CLDN10; |
| chr14 | 101492031 | 101492265 | gene_id MIR323A; transcript_id NR_029890; |
| | | | gene_name MIR323A; |
| chr14 | 104395216 | 104395338 | gene_id TDRD9; transcript_id NM_153046; gene_name |
| | | | TDRD9; |
| chr14 | 33564388 | 33564849 | gene_id NPAS3; transcript_id NM_001164749; |
| | | | gene_name NPAS3; |
| chr14 | 62056738 | 62056968 | gene_id FLJ22447; transcript_id NR_039985; |
| | | | gene_name FLJ22447; |
| chr14 | 90970487 | 90970645 | NA |
| chr14 | 95215739 | 95215812 | NA |
| chr15 | 38653177 | 38653304 | NA |
| chr15 | 49183985 | 49184348 | gene_id SHC4; transcript_id NM_203349; gene_name |
| | | | SHC4; |
| chr15 | 52394089 | 52394248 | NA |
| chr15 | 61181729 | 61181822 | gene_id RORA; transcript_id NM_134261; gene_name |
| | | | RORA; |
| chr15 | 69370689 | 69371017 | NA |
| chr15 | 73674475 | 73674722 | NA |
| chr15 | 99943993 | 99944174 | NA |
| chr16 | 1108660 | 1108765 | NA |
| chr16 | 12084268 | 12084506 | gene_id SNX29; transcript_id NM_001376490; |
| | | | gene_name SNX29; |
| chr16 | 12609458 | 12609724 | gene_id SNX29; transcript_id NM_032167; gene_name |
| | | | SNX29; |
| chr16 | 22937329 | 22937400 | NA |
| chr16 | 56398105 | 56398305 | gene_id AMFR; transcript_id NM_001144; gene_name |
| | | | AMFR; |
| chr16 | 56902235 | 56902745 | gene_id SLC12A3; transcript_id NM_001126108; |
| | | | exon_number 3; exon_id NM_001126108.3; gene_name |
| | | | SLC12A3; |
| chr16 | 66951932 | 66952580 | gene_id CDH16; transcript_id NM_001204745; |
| | | | exon_number 17; exon_id NM_001204745.17; |
| | | | gene_name CDH16; |
| chr16 | 71872180 | 71872562 | NA |
| chr16 | 72038677 | 72039042 | NA |
| chr16 | 74659275 | 74659532 | gene_id RFWD3; transcript_id NM_001370543; |
| | | | gene_name RFWD3; |
| chr16 | 81765201 | 81765593 | NA |
| chr16 | 86523650 | 86523906 | gene_id FENDRR; transcript_id NR_033925; |
| | | | gene_name FENDRR; |
| chr16 | 87693751 | 87693831 | gene_id JPH3; transcript_id NR_073379; gene_name |
| | | | JPH3; |
| chr17 | 1200827 | 1201001 | gene_id TRARG1; transcript_id NM_172367; |
| | | | gene_name TRARG1; |
| chr17 | 1210374 | 1210605 | NA |
| chr17 | 1210620 | 1210755 | NA |
| chr17 | 1299134 | 1299234 | gene_id YWHAE; transcript_id NR_024058; |
| | | | gene_name YWHAE; |
| chr17 | 19618542 | 19618734 | gene_id SLC47A2; transcript_id NM_001099646; |
| | | | gene_name SLC47A2; |
| chr17 | 32956139 | 32956404 | gene_id TMEM132E; transcript_id NM_001304438; |
| | | | exon_number 4; exon_id NM_001304438.4; gene_name |
| | | | TMEM132E; |
| chr17 | 33890350 | 33890582 | NA |
| chr17 | 36967440 | 36967851 | gene_id CWC25; transcript_id NR_073428; gene_name |
| | | | CWC25; |
| chr17 | 47522175 | 47522536 | NA |
| chr17 | 80100626 | 80100870 | gene_id CCDC57; transcript_id NM_001367828; |
| | | | gene_name CCDC57; |
| chr17 | 80645993 | 80646478 | gene_id RAB40B; transcript_id NM_006822; |
| | | | gene_name RAB40B; |
| chr17 | 9800647 | 9800703 | gene_id RCVRN; transcript_id NM_002903; |
| | | | gene_name RCVRN; |
| chr18 | 11992996 | 11993982 | gene_id IMPA2; transcript_id NM_014214; gene_name |
| | | | IMPA2; |
| chr18 | 42290985 | 42291186 | gene_id SETBP1; transcript_id NM_015559; |
| | | | gene_name SETBP1; |
| chr18 | 76151081 | 76151264 | NA |
| chr18 | 76151279 | 76151406 | NA |
| chr18 | 77198244 | 77198384 | gene_id NFATC1; transcript_id NM_006162; |
| | | | gene_name NFATC1; |
| chr19 | 13879658 | 13880041 | gene_id MRI1; transcript_id NM_032285; exon_number |
| | | | 5; exon_id NM_032285.5; gene_name MRI1; |
| chr19 | 14389449 | 14389575 | NA |
| chr19 | 15717940 | 15718120 | NA |
| chr19 | 18595135 | 18595420 | gene_id ELL; transcript_id NM_006532; gene_name |
| | | | ELL; |
| chr19 | 20888866 | 20889154 | NA |
| chr19 | 2233778 | 2233843 | gene_id PLEKHJ1; transcript_id NM_001300836; |
| | | | gene_name PLEKHJ1; |
| chr19 | 31213711 | 31214034 | NA |
| chr19 | 33584939 | 33585374 | gene_id GPATCH1; transcript_id NM_018025; |
| | | | exon_number 5; exon_id NM_018025.5; gene_name |
| | | | GPATCH1; |
| chr19 | 38443593 | 38443789 | gene_id SIPA1L3; transcript_id NM_015073; |
| | | | gene_name SIPA1L3; |
| chr19 | 39180344 | 39180417 | gene_id ACTN4; transcript_id NM_004924; gene_name |
| | | | ACTN4; |
| chr19 | 39529370 | 39529595 | NA |
| chr19 | 54452177 | 54452406 | NA |
| chr19 | 8064907 | 8065003 | gene_id ELAVL1; transcript_id NM_001419; |
| | | | gene_name ELAVL1; |
| chr19 | 968777 | 968944 | gene_id ARID3A; transcript_id NM_005224; |
| | | | gene_name ARID3A; |
| chr2 | 12020617 | 12020905 | NA |
| chr2 | 127882087 | 127882340 | NA |
| chr2 | 127895700 | 127895954 | NA |
| chr2 | 131675628 | 131675775 | gene_id ARHGEF4; transcript_id NM_001367493; |
| | | | gene_name ARHGEF4; |
| chr2 | 148776876 | 148776957 | gene_id ORC4; transcript_id NM_001190881; |
| | | | gene_name ORC4; |
| chr2 | 178017666 | 178017720 | NA |
| chr2 | 178186829 | 178186964 | gene_id LOC100130691; transcript_id NR_026966; |
| | | | gene_name LOC100130691; |
| chr2 | 198948896 | 198949139 | gene_id PLCL1; transcript_id NM_006226; |
| | | | exon_number 2; exon_id NM_006226.2; gene_name |
| | | | PLCL1; |
| chr2 | 203529169 | 203529528 | gene_id FAM117B; transcript_id NM_173511; |
| | | | gene_name FAM117B; |
| chr2 | 209408209 | 209408773 | gene_id LOC101927960; transcript_id NR_136588; |
| | | | gene_name LOC101927960; |
| chr2 | 227191218 | 227191551 | NA |
| chr2 | 228684197 | 228684584 | NA |
| chr2 | 232009704 | 232009997 | gene_id PSMD1; transcript_id NR_034059; gene_name |
| | | | PSMD1; |
| chr2 | 233526562 | 233526619 | gene_id EFHD1; transcript_id NM_025202; gene_name |
| | | | EFHD1; |
| chr2 | 236883965 | 236884437 | gene_id AGAP1; transcript_id NM_001037131; |
| | | | gene_name AGAP1; |
| chr2 | 240722873 | 240723010 | NA |
| chr2 | 240723076 | 240723297 | NA |
| chr2 | 25705734 | 25706207 | gene_id DTNB; transcript_id NM_001351392; |
| | | | exon_number 10; exon_id NM_001351392.10; |
| | | | gene_name DTNB; |
| chr2 | 2995572 | 2995779 | gene_id LINC01250; transcript_id NR_110228; |
| | | | gene_name LINC01250; |
| chr2 | 39015988 | 39016239 | NA |
| chr2 | 43973370 | 43973691 | gene_id PLEKHH2; transcript_id NM_172069; |
| | | | gene_name PLEKHH2; |
| chr2 | 95729375 | 95729636 | NA |
| chr2 | 99302337 | 99302637 | gene_id MGAT4A; transcript_id NM_012214; |
| | | | gene_name MGAT4A; |
| chr20 | 26118583 | 26118678 | NA |
| chr20 | 3371067 | 3371304 | gene_id C20orf194; transcript_id NM_001009984; |
| | | | gene_name C20orf194; |
| chr21 | 26700393 | 26700434 | NA |
| chr21 | 33262156 | 33262645 | gene_id HUNK; transcript_id NM_014586; gene_name |
| | | | HUNK; |
| chr21 | 35212230 | 35212414 | gene_id ITSN1; transcript_id NM_003024; gene_name |
| | | | ITSN1; |
| chr21 | 46666643 | 46666774 | gene_id LINC00334; transcript_id NR_135279; |
| | | | gene_name LINC00334; |
| chr21 | 46848068 | 46848236 | gene_id COL18A1; transcript_id NM_130445; |
| | | | gene_name COL18A1; |
| chr21 | 47049353 | 47049460 | NA |
| chr22 | 32841838 | 32841950 | gene_id BPIFC; transcript_id NM_174932; |
| | | | exon_number 12; exon_id NM_174932.12; gene_name |
| | | | BPIFC; |
| chr22 | 45613514 | 45613840 | gene_id KIAA0930; transcript_id NM_001009880; |
| | | | gene_name KIAA0930; |
| chr22 | 45851255 | 45851438 | NA |
| chr3 | 10485881 | 10485932 | gene_id ATP2B2; transcript_id NM_001353564; |
| | | | gene_name ATP2B2; |
| chr3 | 11111968 | 11112104 | NA |
| chr3 | 125062531 | 125062913 | gene_id ZNF148; transcript_id NM_001348432; |
| | | | gene_name ZNF148; |
| chr3 | 138080099 | 138080567 | gene_id MRAS; transcript_id NM_001252092; |
| | | | gene_name MRAS; |
| chr3 | 153086894 | 153087092 | NA |
| chr3 | 155268258 | 155268686 | gene_id PLCH1; transcript_id NM_001349252; |
| | | | gene_name PLCH1; |
| chr3 | 167925108 | 167925496 | NA |
| chr3 | 192778355 | 192778486 | NA |
| chr3 | 196551996 | 196552204 | gene_id PAK2; transcript_id NM_002577; gene_name |
| | | | PAK2; |
| chr3 | 43261018 | 43261380 | NA |
| chr3 | 46917679 | 46918068 | NA |
| chr3 | 56769039 | 56769267 | gene_id ARHGEF3; transcript_id NM_001128616; |
| | | | gene_name ARHGEF3; |
| chr3 | 86889436 | 86890024 | NA |
| chr3 | 8906619 | 8906866 | NA |
| chr3 | 89480396 | 89480469 | gene_id EPHA3; transcript_id NM_005233; |
| | | | exon_number 13; exon_id NM_005233.13; gene_name |
| | | | EPHA3; |
| chr4 | 116099998 | 116100069 | NA |
| chr4 | 120286506 | 120287047 | NA |
| chr4 | 129735232 | 129735504 | gene_id JADE1; transcript_id NM_001287437; |
| | | | gene_name JADE1; |
| chr4 | 135264651 | 135264883 | NA |
| chr4 | 13877559 | 13878197 | gene_id LINC01182; transcript_id NR_121681; |
| | | | gene_name LINC01182; |
| chr4 | 1534879 | 1535047 | NA |
| chr4 | 187564589 | 187564847 | gene_id FAT1; transcript_id NM_005245; gene_name |
| | | | FAT1; |
| chr4 | 28642378 | 28642648 | NA |
| chr4 | 37984614 | 37984861 | gene_id TBC1D1; transcript_id NM_015173; |
| | | | gene_name TBC1D1; |
| chr4 | 40398957 | 40399177 | NA |
| chr4 | 43719855 | 43719986 | NA |
| chr4 | 73443107 | 73443451 | NA |
| chr4 | 90536238 | 90536686 | NA |
| chr5 | 112953981 | 112954101 | NA |
| chr5 | 130707029 | 130707674 | gene_id CDC42SE2; transcript_id NM_001038702; |
| | | | gene_name CDC42SE2; |
| chr5 | 138122294 | 138123402 | gene_id CTNNA1; transcript_id NM_001323982; |
| | | | gene_name CTNNA1; |
| chr5 | 16681956 | 16682099 | gene_id MYO10; transcript_id NM_012334; |
| | | | exon_number 11; exon_id NM_012334.11; gene_name |
| | | | MYO10; |
| chr5 | 1747254 | 1747335 | NA |
| chr5 | 176148427 | 176148527 | NA |
| chr5 | 178899766 | 178899951 | NA |
| chr5 | 180015927 | 180016082 | NA |
| chr5 | 2237991 | 2238213 | NA |
| chr5 | 40996004 | 40996475 | NA |
| chr5 | 55330568 | 55330870 | NA |
| chr5 | 71714704 | 71714919 | NA |
| chr5 | 73112135 | 73112852 | gene_id ARHGEF28; transcript_id NM_001177693; |
| | | | gene_name ARHGEF28; |
| chr5 | 73115409 | 73115858 | gene_id ARHGEF28; transcript_id NM_001177693; |
| | | | gene_name ARHGEF28; |
| chr5 | 74826137 | 74826703 | gene_id POLK; transcript_id NM_001345921; |
| | | | gene_name POLK; |
| chr5 | 78949976 | 78950127 | gene_id TENT2; transcript_id NM_001297744; |
| | | | gene_name TENT2; |
| chr5 | 79794683 | 79794920 | gene_id FAM151B; transcript_id NM_205548; |
| | | | gene_name FAM151B; |
| chr6 | 1245203 | 1245295 | NA |
| chr6 | 135130051 | 135130252 | NA |
| chr6 | 136188547 | 136189472 | gene_id PDE7B; transcript_id NM_018945; gene_name |
| | | | PDE7B; |
| chr6 | 149868192 | 149868429 | NA |
| chr6 | 153471499 | 153471660 | NA |
| chr6 | 153471717 | 153471870 | NA |
| chr6 | 154797522 | 154797579 | gene_id CNKSR3; transcript_id NM_001368118; |
| | | | gene_name CNKSR3; |
| chr6 | 15525028 | 15525518 | gene_id DTNBP1; transcript_id NM_032122; |
| | | | gene_name DTNBP1; |
| chr6 | 170564874 | 170565152 | gene_id LOC154449; transcript_id NR_002787; |
| | | | gene_name LOC154449; |
| chr6 | 21208776 | 21209060 | gene_id CDKAL1; transcript_id NM_017774; |
| | | | gene_name CDKAL1; |
| chr6 | 241052 | 241154 | NA |
| chr6 | 443962 | 444098 | NA |
| chr6 | 4468938 | 4469316 | NA |
| chr6 | 6637390 | 6637534 | gene_id LY86; transcript_id NM_004271; gene_name |
| | | | LY86; |
| chr6 | 93438195 | 93438327 | NA |
| chr6 | 96084977 | 96085304 | NA |
| chr7 | 12583963 | 12584063 | NA |
| chr7 | 129688596 | 129689387 | gene_id ZC3HC1; transcript_id NM_001363701; |
| | | | exon_number 9; exon_id NM_001363701.9; gene_name |
| | | | ZC3HC1; |
| chr7 | 150684071 | 150684260 | NA |
| chr7 | 150684315 | 150684378 | NA |
| chr7 | 151151464 | 151151679 | NA |
| chr7 | 151781437 | 151781869 | gene_id GALNT11; transcript_id NM_022087; |
| | | | gene_name GALNT11; |
| chr7 | 154656077 | 154656713 | gene_id DPP6; transcript_id NM_001364500; |
| | | | gene_name DPP6; |
| chr7 | 156883572 | 156883759 | NA |
| chr7 | 157280199 | 157280369 | gene_id LOC101927914; transcript_id NR_110157; |
| | | | gene_name LOC101927914; |
| chr7 | 157427089 | 157427365 | gene_id PTPRN2; transcript_id NM_001308268; |
| | | | gene_name PTPRN2; |
| chr7 | 46185466 | 46185603 | NA |
| chr7 | 5886722 | 5886892 | gene_id ZNF815P; transcript_id NR_023382; |
| | | | exon_number 5; exon_id NR_023382.5; gene_name |
| | | | ZNF815P; |
| chr7 | 68500931 | 68500967 | NA |
| chr7 | 68740251 | 68740436 | NA |
| chr7 | 8175827 | 8176181 | gene_id ICA1; transcript_id NM_001136020; |
| | | | gene_name ICA1; |
| chr7 | 87640622 | 87640842 | gene_id ADAM22; transcript_id NM_004194; |
| | | | gene_name ADAM22; |
| chr8 | 1132771 | 1132948 | gene_id DLGAP2; transcript_id NM_001346810; |
| | | | gene_name DLGAP2; |
| chr8 | 116559924 | 116560157 | gene_id TRPS1; transcript_id NM_001282903; |
| | | | gene_name TRPS1; |
| chr8 | 144017883 | 144018008 | NA |
| chr8 | 58004260 | 58004438 | NA |
| chr8 | 60913221 | 60913422 | NA |
| chr8 | 68285283 | 68285609 | gene_id LOC102724708; transcript_id NR_136223; |
| | | | gene_name LOC102724708; |
| chr8 | 68323403 | 68323481 | gene_id LOC102724708; transcript_id NR_136224; |
| | | | gene_name LOC102724708; |
| chr8 | 75091047 | 75091117 | NA |
| chr9 | 134719922 | 134720096 | NA |
| chr9 | 140901257 | 140901499 | gene_id CACNA1B; transcript_id NM_000718; |
| | | | exon_number 16; exon_id NM_000718.16; gene_name |
| | | | CACNA1B; |
| chr9 | 45737530 | 45738006 | NA |
| chr9 | 46121596 | 46121710 | NA |
| chr9 | 4756288 | 4756435 | NA |
| chr9 | 85557951 | 85558014 | NA |
| chr9 | 92053603 | 92053682 | gene_id SEMA4D; transcript_id NM_001371201; |
| | | | gene_name SEMA4D; |
| chr9 | 92053737 | 92053936 | gene_id SEMA4D; transcript_id NM_001371201; |
| | | | gene_name SEMA4D; |
| chr9 | 97412072 | 97412253 | NA |
| chr9 | 97610227 | 97610861 | gene_id AOPEP; transcript_id NM_001193329; |
| | | | gene_name AOPEP; |
| chrY | 16487057 | 16487297 | NA |
| chrY | 2830422 | 2831112 | gene_id ZFY; transcript_id NM_001145276; |
| | | | gene_name ZFY; |

Step 3: Assign cfDNA Reads or Fragments to a Cell Type or Tissue of Origin Based on its Read-Level or Fragment-Level Methylation

To assign cfDNA reads or fragments to a cell type or tissue of origin, only reads mapped to the cell type or tissue-specific marker regions will be kept for analysis. The unmethylated reads will be considered as derived from cfDNA released from the corresponding cell type or tissue. However, due to the complexity of cfDNA release and clearance, as well as multiple potential biases from cfDNA extraction, profiling and methylation measurement, we have developed 4 strategies to normalize and quantify the cell type or tissue-specific DNA. Among them, only method 2 and method 4 can be applied to targeted sequencing data. All of these 4 methods can be applied to whole genome methylation profiling data. As shown in FIG. 12, if reads mapped to region Lᵢ, which is marker region for tissue/cell type i, then both unmethylated (uᵢ) and methylated (mᵢ) reads mapped to the region Lᵢ will be taken into consideration for calculation. Details about these methods are illustrated as follows:

### 6.2.2 Method 1: Relative Cell Type Fraction Across all Marker Regions

Raw fraction (pᵢ) of a specific cell type was calculated as the ratio of the unmethylated reads (uᵢ) within the cell type-specific marker regions (Lᵢ) to the sum of all unmethylated reads across all marker regions for all cell types or tissues included in the reference panel (U, which is the sum of all uᵢ, where i ranges from 1 to n). Then, the raw fraction was normalized by dividing the sum of all cell types to get the relative proportion of each cell type. This method is denoted as “Ratio_AcrossReference”. This method cannot be applied to targeted sequencing datasets.

6.2.3 Method 2: Relative Cell Type Fraction within Corresponding Marker Regions

Raw fraction of a specific cell type was calculated as the ratio of the unmethylated reads (uᵢ) to all the reads within the corresponding cell type-specific marker regions (Tᵢ, which is the sum of uᵢ and mᵢ for the i^{th} marker region corresponding to tissue i). Then, the raw fraction is normalized by dividing the sum of all cell types to get the relative proportion of each cell type. This method is denoted as “Ratio_WithinReference”. This method can be applied to targeted sequencing datasets. For example, based on our panel targeting kidney-specific marker regions including 250 genomic sites, the raw fraction will be calculated as the ratio of unmethylated reads (uᵢ) mapped within the 250 genomic sites to all the reads mapped within the 250 genomic sites. No scaling is required.

### 6.2.4 Method 3: Reads Abundance Normalized by Sequencing Depth Across all Marker Regions

This method is mainly to normalize the sequencing depth's effect on the quantification. Read per kilobases per million (RPKM) value of a specific cell type i is calculated as the unmethylated reads (uᵢ) within the cell type-specific marker regions and normalized by the length of marker regions (Lᵢ) and total reads across all marker regions (T, which is the sum of all uⱼ and mᵢ, where i ranges from 1 to n). This method is denoted as “RPKM_AcrossReference”. This method cannot be applied to targeted sequencing datasets.

### 6.2.5 Method 4: Reads Abundance Normalized by Sequencing Depth at Specific Marker Region

This method is mainly to normalize the sequencing depth's effect on the quantification. RPKM value of a specific cell type i was calculated as the unmethylated reads (uᵢ) normalized by the length of corresponding marker regions (Lᵢ) and the total reads within the cell type-specific marker regions (Tᵢ, which is the sum of uⱼ and mᵢ for the i^{th} marker region corresponding to tissue i). This method is denoted as “RPKM_WithinReference”. This method can be applied to targeted sequencing datasets. For example, based on the panel targeting kidney-specific marker regions including 250 genomic sites, RPKM value of the kidney is calculated as the unmethylated reads (uᵢ) mapped within the 250 genomic sites normalized by the total length of marker regions (around 64 kb in total) and the total reads mapped to the 250 genomic sites.

Step 4: Determine Cell Type or Tissue-Specific cfDNA Level as a Reflection of Potential Cell or Tissue Damage

After quantifying the cell type or tissue-specific cfDNA level based on reads ratio or RPKM, the results will be compared to a threshold range to determine whether the level is abnormally high or not. The abnormally high level of cell type or tissue-specific cfDNA level indicates damages of the specific tissue. In our SLE patient cohort, we have compared the level of kidney cfDNA between active lupus nephritis patients and other SLE patients, as well as to healthy donors. From the comparison, the cutoff range from these 4 methods are as follows:

| | Normal value range | High risk of kidney |
|---|---|---|
| Normalization method | (kidney) | damage |
| Ratio_AcrossReference | <0.017 | >=0.020 |
| Ratio_WithinReference | <0.015 | >=0.015 |
| RPKM_AcrossReference | <10 | >=12 |
| RPKM_WithinReference | <390 | >=400 |

Kidney damage will be determined if the level of kidney cfDNA is above the normal range for different methods indicated in the table above. 6.3 Statistical analysis

Kruskal-Wallis test was applied for the comparison among multiple groups, and the Wilcoxon rank sum test was applied for 2-group comparison if not specifically stated. Spearman coefficients were calculated for correlation analysis. All statistical analysis and visualization was performed in R (version 4.0.3).

### 6.3 Results

### 6.3.1 Custom Methods Outperform Conventional NNLS Method for Deconvolution of Cell Type Origin

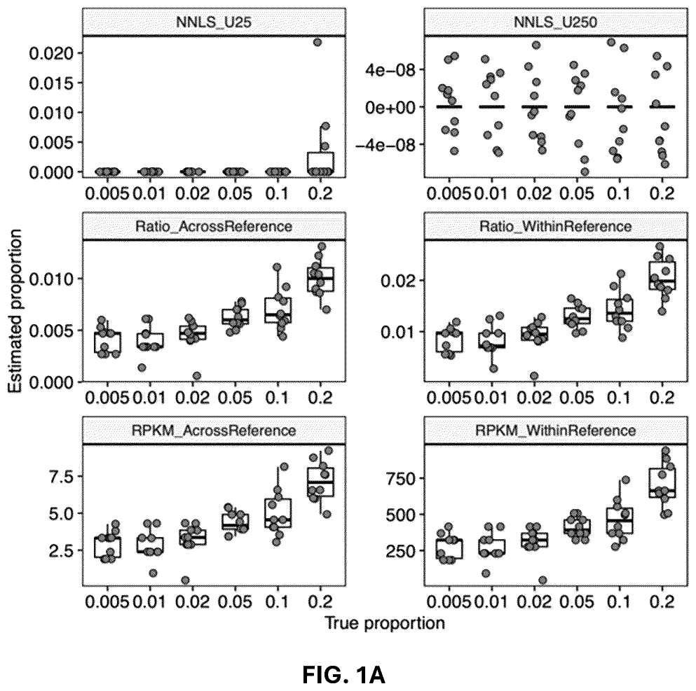

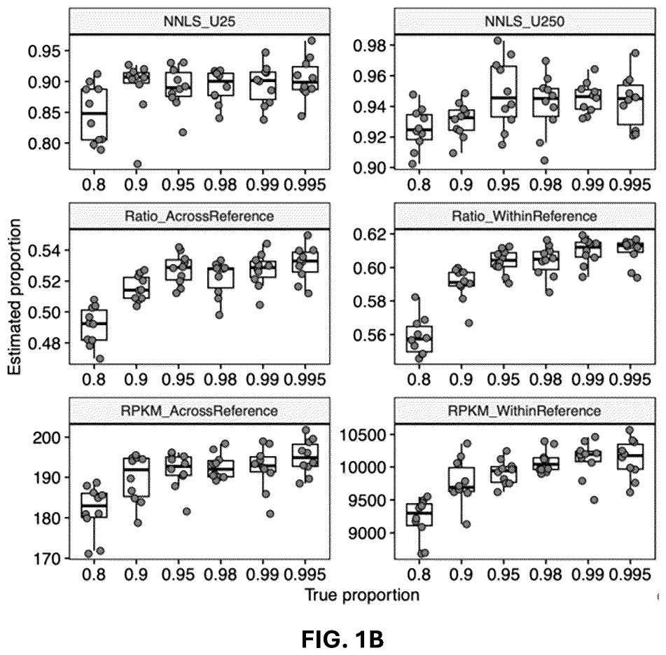

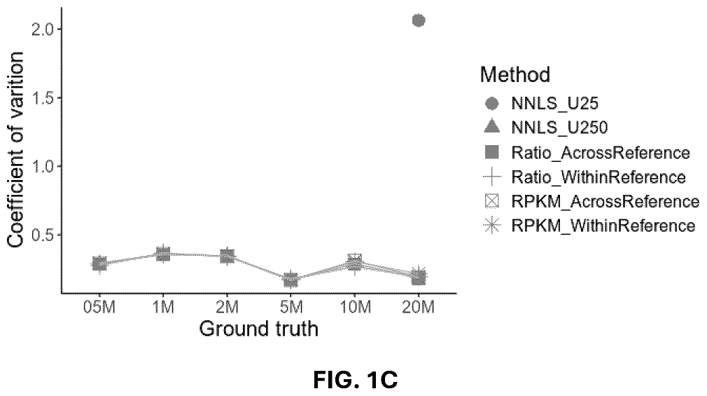

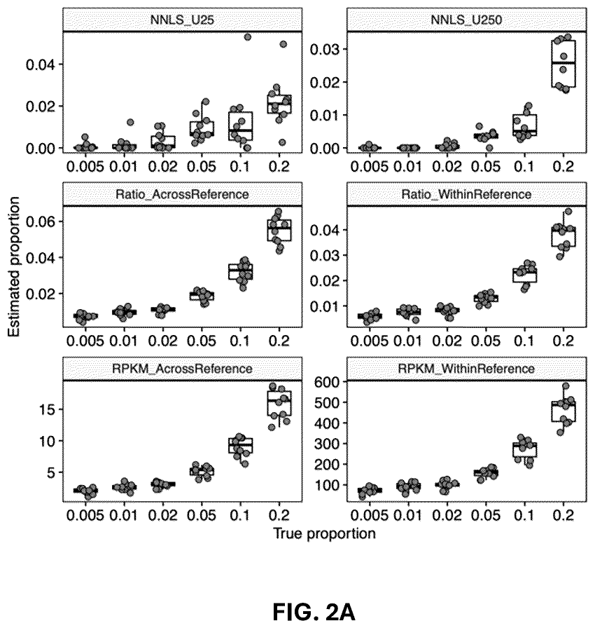

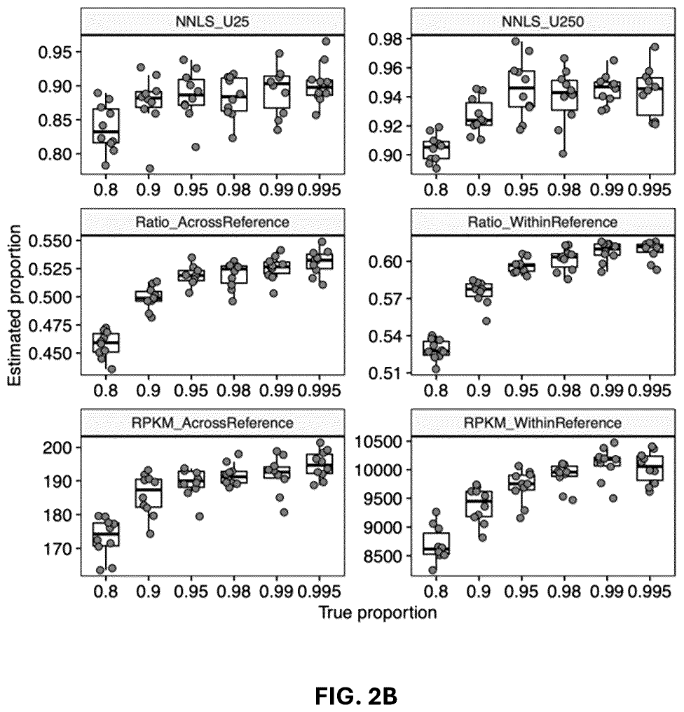

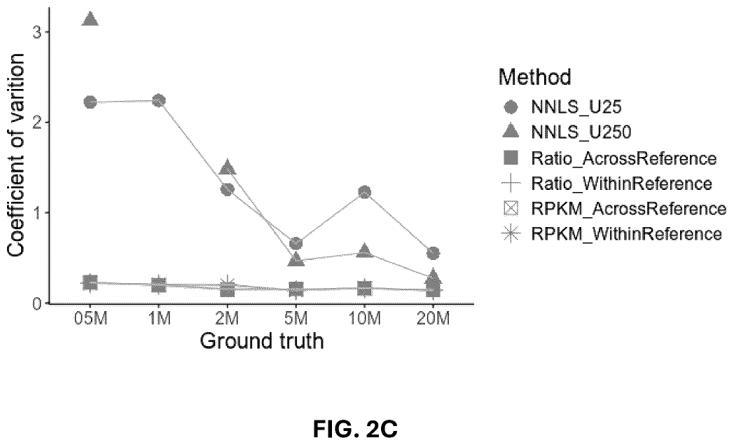

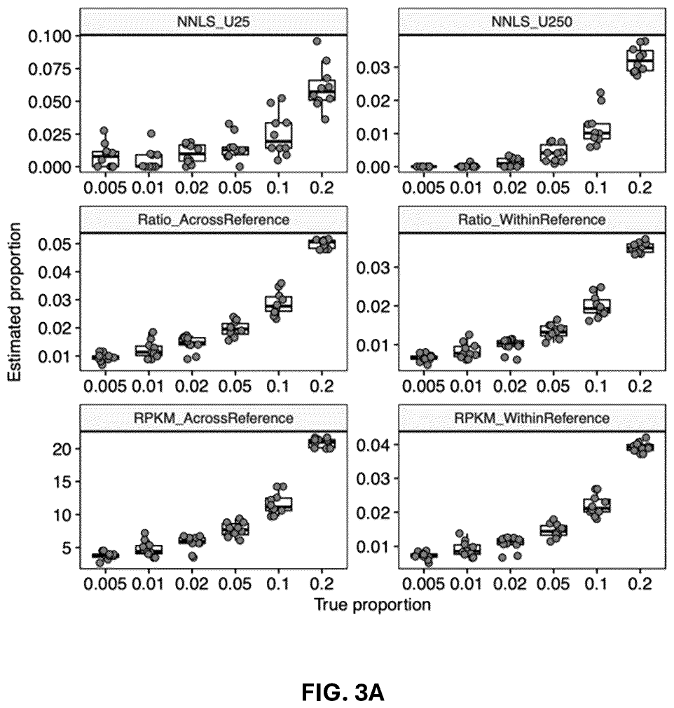

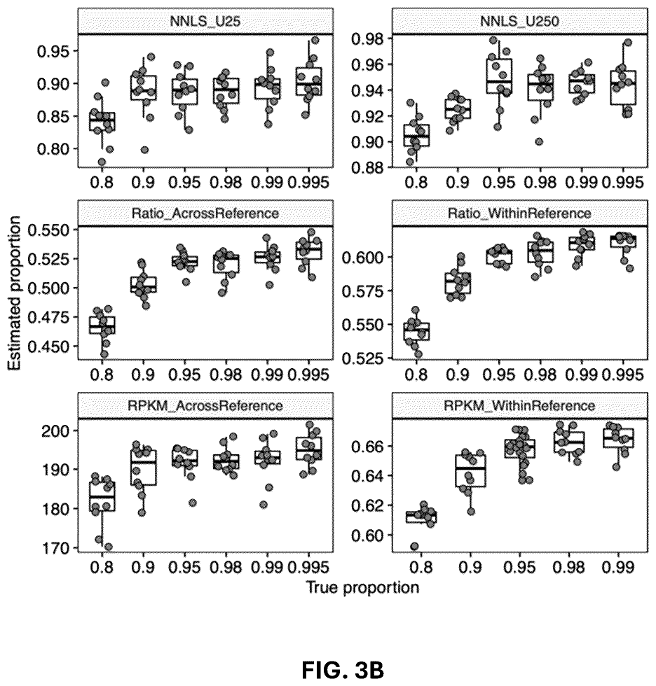

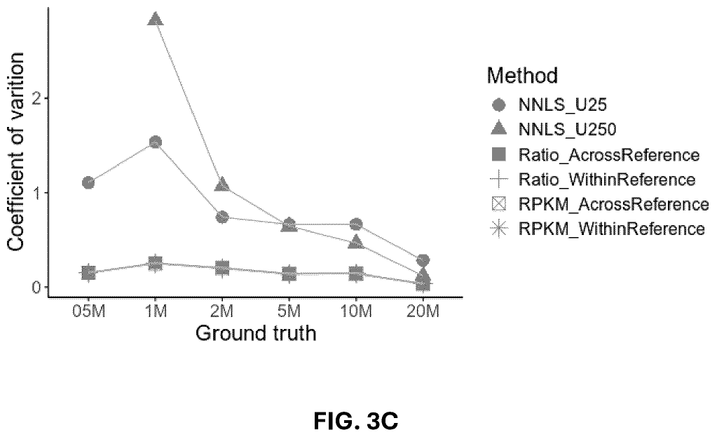

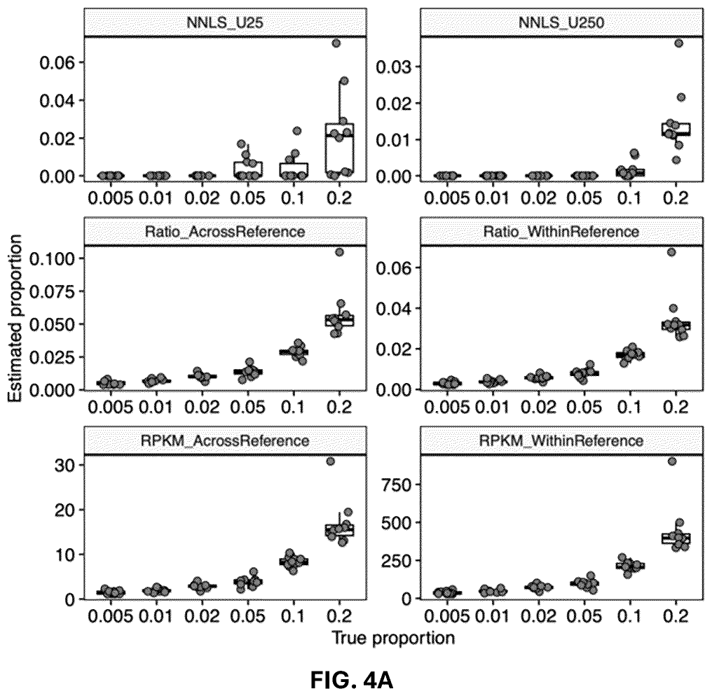

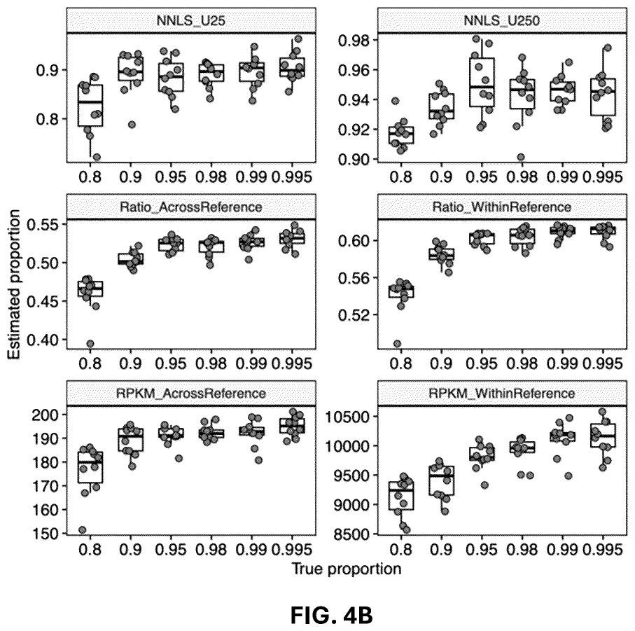

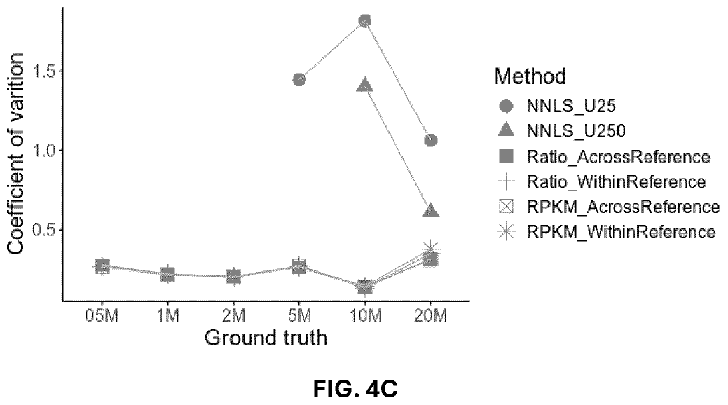

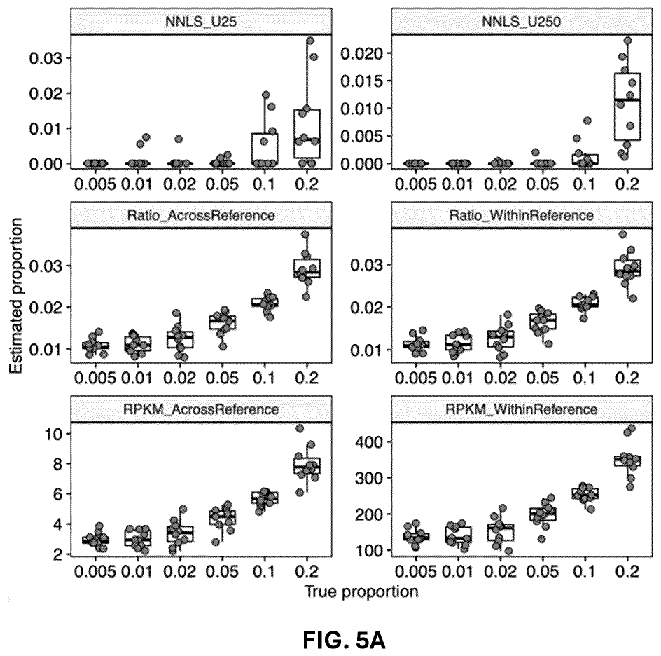

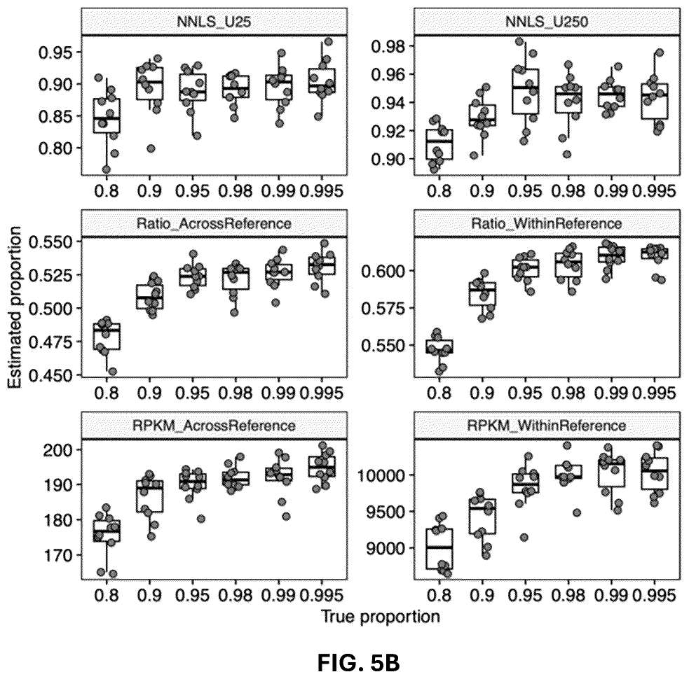

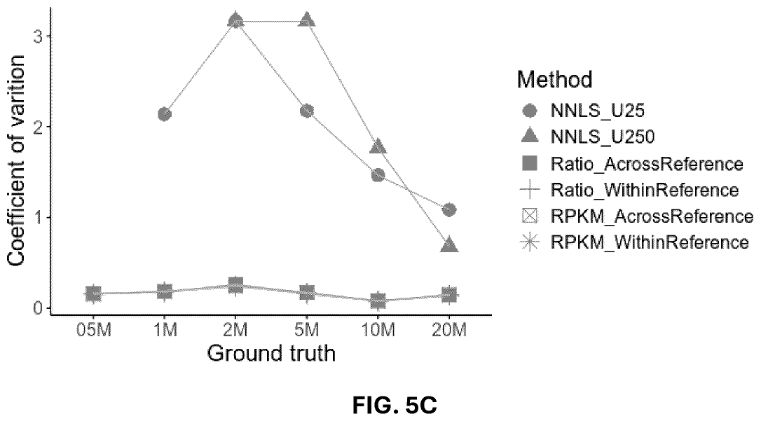

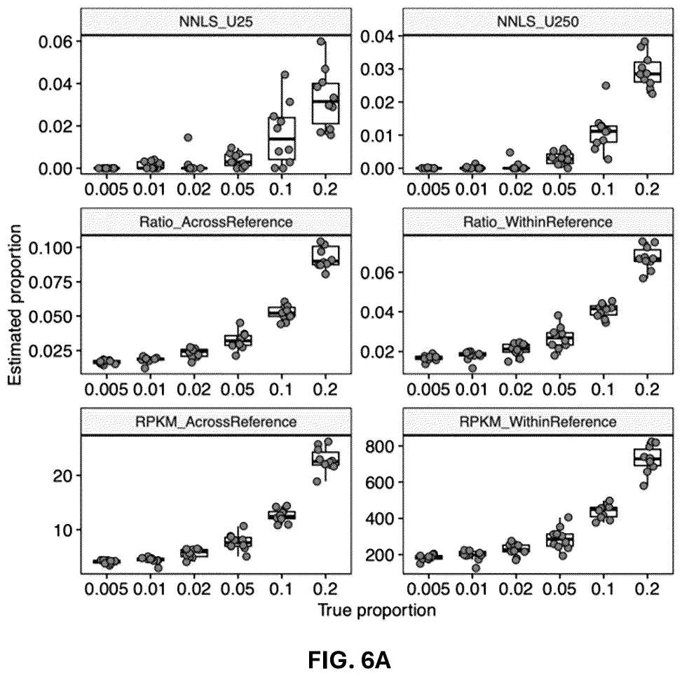

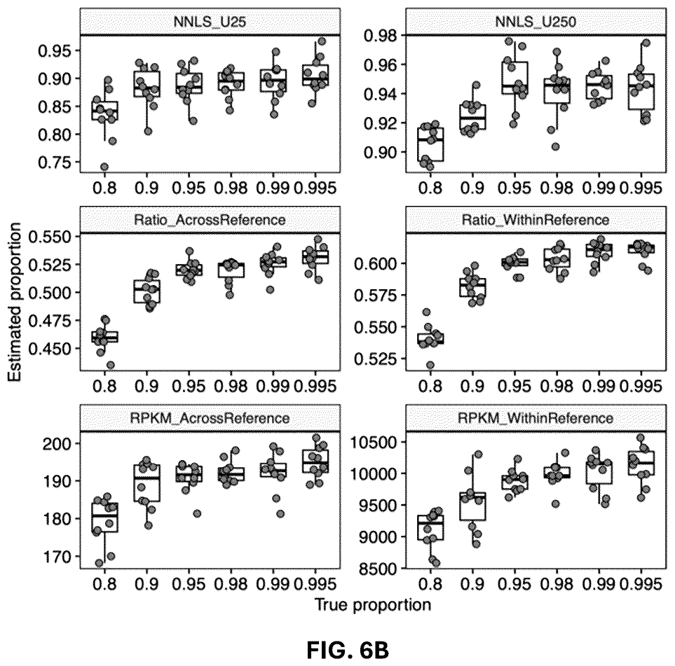

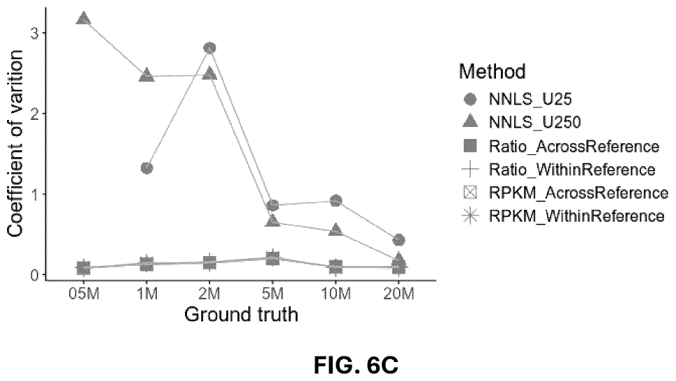

Blood cells are the major contributors to human blood cfDNA, with granulocytes, monocytes/macrophages and megakaryocytes occupying more than 90% of cfDNA 10.11. To test the performance of the custom method, whole-genome bisulfite sequencing data were simulated by mixing different tissues with monocytes to mimic cfDNA WGBS data. The proportion of targeted tissues are 0.5%, 1%, 2%, 5%, 10% and 20% respectively to mimic the low abundance of non-blood originated cfDNA. WGBS data from adipose, heart, kidney, liver, lung and pancreas were included in the simulation. Deconvolution was then performed using both the present methods and conventional NNLS method with top 25 and top 250 marker regions for each cell type. As shown in FIG. 1-6, the present methods outperform NNLS to profile cell type components with low abundance, especially for adipose, liver and lung, where conventional NNLS cannot detect targeted tissues with abundance below 5%. Even for the monocytes, which has high abundance in all simulated datasets, the present methods reflect better of the different abundance level. Moreover, the present methods also demonstrate much lower within-group variability for all the simulations, indicating the robustness of these methods.

6.3.2 Kidney cfDNA as Biomarker for Lupus Nephritis (LN)

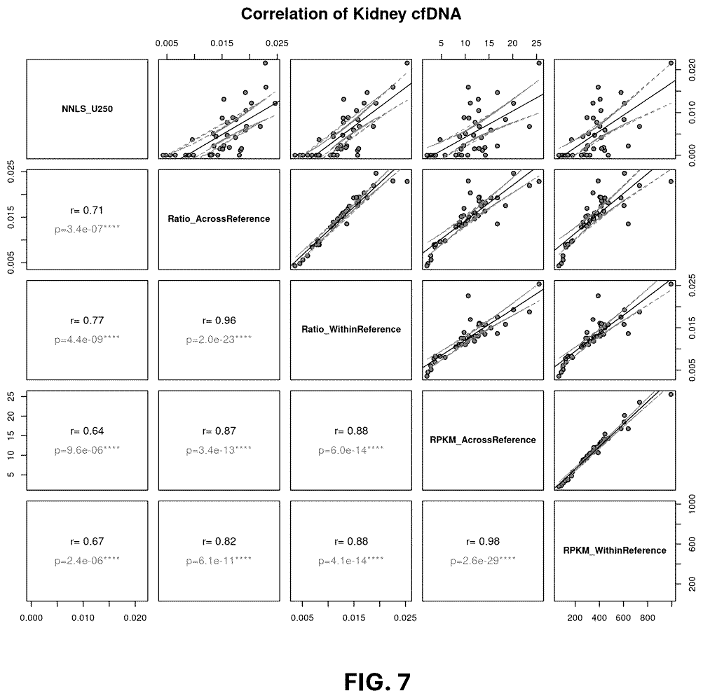

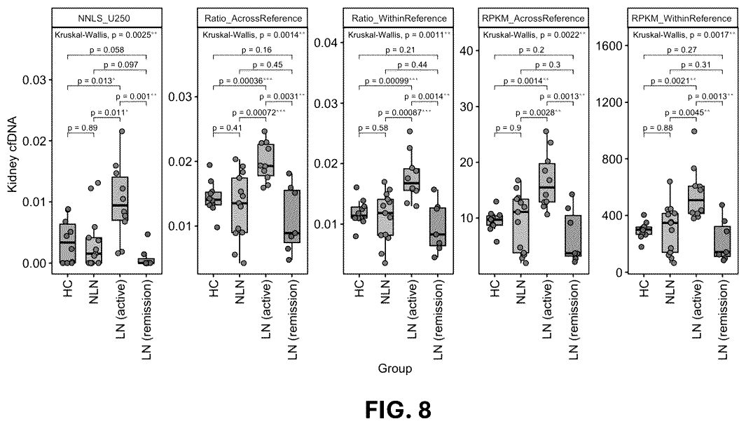

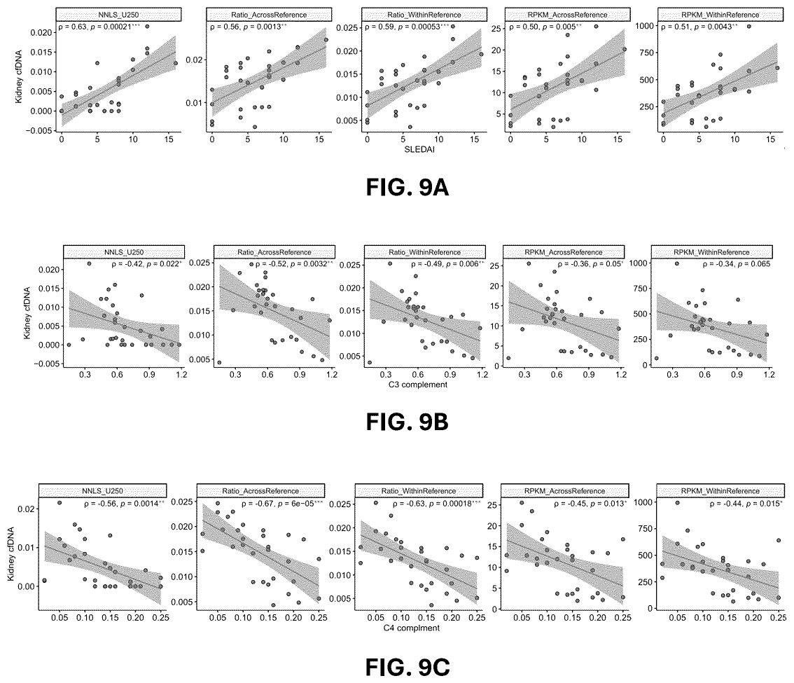

Nephritis is one of the most severe manifestations of systemic lupus erythematosus (SLE) ^{17,18}. Clinically, kidney biopsy is the gold standard for diagnosis of nephritis diagnosis, which is highly invasive and risky. Non-invasive biomarkers for nephritis diagnosis and monitoring are lacking^{19,20}. To further demonstrate the application of the present methods, SLE patients with and without active lupus nephritis (LN) were recruited and their blood cfDNA methylation was profiled. Again, both custom methods and NNLS were implied (with top 250 marker regions) to identify kidney-derived cfDNA components. In general, the results from NNLS and the present methods are comparable. Consistent with the simulated data, the present methods are more sensitive to profile kidney cfDNA with low abundance (FIG. 7). Significantly higher kidney cfDNA was also observed in patients with active nephritis compared to healthy donors, non-LN SLE patients and remission LN patients (FIG. 8). Moreover, kidney cfDNA is positively correlated with SLEDAI score and negatively correlated with blood level of C3 and C4 complements (FIG. 9).

### 6.4 Discussion

One advantage of the DNA methylation signal is that the tissue or cell type origin of cfDNA can be traced in liquid biopsy samples, such as blood, urine and so on, which makes the measurement much easier and moreover enables one to detect the involvement of different tissues or cell types. Currently, there are some commonly used methods to measure the cfDNA methylation signals, including whole-genome bisulfite sequencing, EM-Seq, RRBS-seq, Methylation Array. These methods provide a general DNA methylation profiling. A key point is how to deconvolute the methylation signal and thus to untangle the composition of cfDNA. As described in herein, the present application provides a new and easy method for this purpose. Compared to previous methods, the biggest difference is that the present methods does not need to compare the cfDNA methylation profile to the reference methylation signatures. Rather, the present methods can directly profile the cfDNA methylation signals at the tissue-of-interest specific regions to calculate the abundance of that tissue in a facile way. This strategy can make it possible to measure the abundance of tissue-of-interest by target sequencing of specific regions and as such reduce the cost and turnaround time.

### Exemplary Products, Systems and Methods are Set Out in the Following Items:

1. A method of treating a subject having a cell, tissue or organ damage from a disease or disorder, the method comprising: (a) obtaining a sample from the subject containing cfDNA; (b) receiving a plurality of read-level or fragment-level cell-free deoxyribonucleic acid (“cfDNA”) methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; (e) determining the reads ratio or RPKM as compared to a control sample from a subject without the cell or tissue damage, wherein the reads ratio or RPKM above a threshold range indicates that the subject has damage in the specific cell-type or tissue; and (f) administering a treatment to the subject based on the identifying the subject as having the disorder.

2. The method of item 1 wherein the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

3. The method of item 1 or 2 wherein the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

4. The method of any one of items 1-3 wherein the diseases or disorder indicates a cell, organ and tissue damage from graft rejections in organ transplantation, immune related diseases, lupus nephritis, myocarditis, infection, cancer and radiation induced damages.

5. The method of any one of items 1-4, wherein the cfDNA sample is obtained or derived from a plasma sample, a blood sample, a saliva sample, an amniotic fluid sample, a cystic fluid sample, a spinal fluid sample, a brain fluid sample, a urine sample, a sweat sample, or a tears sample from the subject.

6. The method of any one of items 1-5, wherein the organ or tissue is adipose, bladder, colon, skin, stomach, heart, kidney, liver, lung, brain, pancreas, prostate, small intestine, thyroid or a combination thereof.

7. The method of any one of items 1-6, wherein the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

8. The method of any one of items 1-7, wherein the treatment is chemotherapy, radiation therapy, immunotherapy, target therapy and tumor resection.

9. The method of any one of items 1-8, wherein the diseases or disorder indicates a cell, organ and tissue damage from graft rejections in organ transplantation, immune related diseases, lupus nephritis, myocarditis, infection, cancer and radiation induced damages.

10. A method of identifying tissue-specific damage in a subject having a disease or disorder, comprising: (a) receiving a plurality of sequencing reads for a cell-free deoxyribonucleic acid (cfDNA) sample obtained or derived from the subject, wherein each of the plurality of sequencing reads comprises methylation sequencing data obtained from a nucleic acid sequence; (b) determining a methylation pattern for a sequencing read in the plurality of sequencing reads, wherein the methylation pattern comprises a genomic region corresponding to the nucleic acid sequence and methylation status of one or more motifs in the genomic region; (c) characterizing the cfDNA sample as containing cfDNAs derived from a tissue of the subject based on a reads ratio or RPKM, wherein the characterization of the cfDNA as being derived from the tissue of the subject indicates tissue-specific damage to the tissue of the subject.

11. The method of item 10 wherein the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

12. The method of item 10 or 11 wherein the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

13. The method of any one of items 10-12 wherein the diseases or disorder indicates a cell, organ and tissue damage from graft rejections in organ transplantation, immune related diseases, lupus nephritis, myocarditis, infection, cancer and radiation induced damages.

14. The method of any one of items 10-13, wherein the cfDNA sample is obtained or derived from a plasma sample, a blood sample, a saliva sample, an amniotic fluid sample, a cystic fluid sample, a spinal fluid sample, a brain fluid sample, a urine sample, a sweat sample, or a tears sample from the subject.

15. The method of any one of items 10-14, wherein the organ or tissue is adipose, bladder, colon, skin, stomach, heart, kidney, liver, lung, brain, pancreas, prostate, small intestine, thyroid or a combination thereof.

16. The method of any one of items 10-15, wherein the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

17. The method of any one of items 10-16, further comprising a step of treating the subject with chemotherapy, radiation therapy, immunotherapy, target therapy, tumor resection or a combination thereof.

18. The method of any one of items 10-17, wherein the diseases or disorder indicates a cell, organ and tissue damage from graft rejections in organ transplantation, immune related diseases, lupus nephritis, myocarditis, infection, cancer and radiation induced damages.

19. A method of monitoring a subject having a cell or tissue or organ damage from a disease or disorder after treatment, wherein the monitoring comprises at least two times, the method comprising: (a) obtaining a sample from the subject containing cfDNA; (b) receiving a plurality of read-level or fragment-level cell-free deoxyribonucleic acid (“cfDNA”) methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; (c) determining the reads ratio or RPKM as compared to a control sample from a subject without the cell or tissue damage, wherein the reads ratio or RPKM having an above threshold range prior to a first treatment indicates that the subject has damage in the specific cell-type or tissue, and wherein the reads ratio or RPKM after a first time point after a first treatment having a below threshold range indicates the treatment is effective, and wherein the reads ratio or RPKM after a second time point after the first treatment having a higher threshold range indicates that the treatment is effective at the first time point but recurred at the second time point.

20. The method of item 19 wherein the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

21. The method of item 19 or 20 wherein the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

22. The method of any one of items 19-21 wherein the disorder could be varieties of organ and tissue involved pathological conditions, including but not limited to graft rejections in organ transplantations, immune related diseases such as lupus nephritis and myocarditis, infection, cancer and radiation induced tissue damages of the subject.

23. The method of any of items 19-22, wherein the cfDNA sample is obtained or derived from body fluids, including but not limited to a plasma sample, a blood sample, a saliva sample, an amniotic fluid sample, a cystic fluid sample, a spinal fluid sample, a brain fluid sample, a urine sample, a sweat sample, or a tears sample from the subject.

24. The method of any one of items 19-23, wherein the organ or tissue is adipose, bladder, colon, skin, stomach, heart, kidney, liver, lung, brain, pancreas, prostate, small intestine, thyroid or a combination thereof.

25. The method of any one of items 19-24, wherein the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

26. The method of any one of items 19-25, wherein the treatment is chemotherapy, radiation therapy, immunotherapy, target therapy and tumor resection.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Fan, H. C., Blumenfeld, Y. J., Chitkara, U., Hudgins, L. & Quake, S. R. Noninvasive diagnosis of fetal aneuploidy by shotgun sequencing DNA from maternal blood. Proc. Natl. Acad. Sci. U.S.A 105, 16266-16271 (2008).
2. Chiu, R. W. K. et al. Noninvasive prenatal diagnosis of fetal chromosomal aneuploidy by massively parallel genomic sequencing of DNA in maternal plasma. Proc. Natl. Acad. Sci. 105, 20458-20463 (2008).
3. Snyder, T. M., Khush, K. K., Valantine, H. A. & Quake, S. R. Universal noninvasive detection of solid organ transplant rejection. Proc. Natl. Acad. Sci. 108, 6229-6234 (2011).
4. De Vlaminck, I. et al. Noninvasive monitoring of infection and rejection after lung transplantation. Proc. Natl. Acad. Sci. U.S.A 112, 13336-13341 (2015).
5. Knight, S. R., Thorne, A. & Lo Faro, M. L. Donor-specific Cell-free DNA as a Biomarker in Solid Organ Transplantation. A Systematic Review. Transplantation 103, (2019).
6. Allen, C. K. C. et al. Analysis of Plasma Epstein-Barr Virus DNA to Screen for Nasopharyngeal Cancer. N. Engl. J. Med. 377, 513-522 (2024).
7. C., C. D. et al. A Cell-free DNA Blood-Based Test for Colorectal Cancer Screening. N. Engl. J. Med. 390, 973-983 (2024).
8. Kobayashi, Y. et al. DNA methylation profiling reveals novel biomarkers and important roles for DNA methyltransferases in prostate cancer. Genome Res. 21, 1017-1027 (2011).
9. Moss, J. et al. Comprehensive human cell-type methylation atlas reveals origins of circulating cell-free DNA in health and disease. Nat. Commun. 9, (2018).
10. Loyfer, N. et al. A DNA methylation atlas of normal human cell types. Nature 613, 355-364 (2023).
11. Cheng, A. P. et al. Cell-free DNA tissues of origin by methylation profiling reveals significant cell, tissue, and organ-specific injury related to COVID-19 severity. Med 2, 411-422.e5 (2021).
12. Caggiano, C. et al. Comprehensive cell type decomposition of circulating cell-free DNA with CelFiE. Nat. Commun. 12, 2717 (2021).
13. De Ridder, K., Che, H., Leroy, K. & Thienpont, B. Benchmarking of methods for DNA methylome deconvolution. Nat. Commun. 15, 4134 (2024).
14. Chan, R. W. Y. et al. Plasma DNA aberrations in systemic lupus erythematosus revealed by genomic and methylomic sequencing. Proc. Natl. Acad. Sci. 111, E5302-E5311 (2014).
15. Krueger, F. & Andrews, S. R. Bismark: a flexible aligner and methylation caller for Bisulfite-Seq applications. Bioinformatics 27, 1571-1572 (2011).
16. Hetzel, S., Giesselmann, P., Reinert, K., Meissner, A. & Kretzmer, H. RLM: fast and simplified extraction of read-level methylation metrics from bisulfite sequencing data. Bioinformatics 37, 3934-3935 (2021).
17. Danila, M. I. et al. Renal damage is the most important predictor of mortality within the damage index: data from LUMINA LXIV, a multiethnic US cohort. Rheumatology 48, 542-545 (2009).
18. Wilson, H. R. & Lightstone, L. Manifestations of lupus in the kidney and how to manage them. Nephrol. Dial. Transplant. 32, 1614-1616 (2017).
19. Soliman, S. & Mohan, C. Lupus nephritis biomarkers. Clin. Immunol. 185, 10-20 (2017).
20. Anders, H.-J. et al. Lupus nephritis. Nat. Rev. Dis. Prim. 6, 7 (2020).
21. Loyfer et al., Nature 2023; doi: 10.1038/s41586-022-05580-6.

What is claimed:

1. A method of treating a subject having a cell, tissue or organ damage from a disease or disorder, the method comprising: (a) obtaining a sample from the subject containing cfDNA; (b) receiving a plurality of read-level or fragment-level cell-free deoxyribonucleic acid (“cfDNA”) methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; (e) determining the reads ratio or RPKM as compared to a control sample from a subject without the cell or tissue damage, wherein the reads ratio or RPKM above a threshold range indicates that the subject has damage in the specific cell-type or tissue; and (f) administering a treatment to the subject based on the identifying the subject as having the disorder.

2. The method of claim 1 wherein the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

3. The method of claim 1 wherein the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

4. The method of claim 1 wherein the diseases or disorder indicates a cell, organ and tissue damage from graft rejections in organ transplantation, immune related diseases, lupus nephritis, myocarditis, infection, cancer and radiation induced damages.

5. The method of claim 1, wherein the cfDNA sample is obtained or derived from a plasma sample, a blood sample, a saliva sample, an amniotic fluid sample, a cystic fluid sample, a spinal fluid sample, a brain fluid sample, a urine sample, a sweat sample, or a tears sample from the subject.

6. The method of claim 1, wherein the organ or tissue is adipose, bladder, colon, skin, stomach, heart, kidney, liver, lung, brain, pancreas, prostate, small intestine, thyroid or a combination thereof.

7. The method of claim 1, wherein the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

8. The method of claim 7, wherein the treatment is chemotherapy, radiation therapy, immunotherapy, target therapy and tumor resection.

9. The method of claim 1, wherein the diseases or disorder indicates a cell, organ and tissue damage from graft rejections in organ transplantation, immune related diseases, lupus nephritis, myocarditis, infection, cancer and radiation induced damages.

10. A method of identifying tissue-specific damage in a subject having a disease or disorder, comprising: (a) receiving a plurality of sequencing reads for a cell-free deoxyribonucleic acid (cfDNA) sample obtained or derived from the subject, wherein each of the plurality of sequencing reads comprises methylation sequencing data obtained from a nucleic acid sequence; (b) determining a methylation pattern for a sequencing read in the plurality of sequencing reads, wherein the methylation pattern comprises a genomic region corresponding to the nucleic acid sequence and methylation status of one or more motifs in the genomic region; (c) characterizing the cfDNA sample as containing cfDNAs derived from a tissue of the subject based on a reads ratio or RPKM, wherein the characterization of the cfDNA as being derived from the tissue of the subject indicates tissue-specific damage to the tissue of the subject.

11. The method of claim 10 wherein the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

12. The method of claim 10 wherein the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

13. The method of claim 10 wherein the diseases or disorder indicates a cell, organ and tissue damage from graft rejections in organ transplantation, immune related diseases, lupus nephritis, myocarditis, infection, cancer and radiation induced damages.

14. The method of claim 10, wherein the cfDNA sample is obtained or derived from a plasma sample, a blood sample, a saliva sample, an amniotic fluid sample, a cystic fluid sample, a spinal fluid sample, a brain fluid sample, a urine sample, a sweat sample, or a tears sample from the subject.

15. The method of claim 10, wherein the organ or tissue is adipose, bladder, colon, skin, stomach, heart, kidney, liver, lung, brain, pancreas, prostate, small intestine, thyroid or a combination thereof.

16. The method of claim 10, wherein the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

17. The method of claim 10, further comprising a step of treating the subject with chemotherapy, radiation therapy, immunotherapy, target therapy, tumor resection or a combination thereof.

18. The method of claim 10, wherein the diseases or disorder indicates a cell, organ and tissue damage from graft rejections in organ transplantation, immune related diseases, lupus nephritis, myocarditis, infection, cancer and radiation induced damages.

19. A method of monitoring a subject having a cell or tissue or organ damage from a disease or disorder after treatment, wherein the monitoring comprises at least two times, the method comprising: (a) obtaining a sample from the subject containing cfDNA; (b) receiving a plurality of read-level or fragment-level cell-free deoxyribonucleic acid (“cfDNA”) methylation sequencing reads, wherein each of the plurality of sequencing reads comprises methylation sequencing data corresponding to a genomic region that is cell-type specific or tissue specific; (c) normalizing the read-level or fragment level cfDNA methylation sequencing reads that is cell-type specific or tissue-specific; (d) assigning the read-level or fragment-level cfDNA methylation to the cell-type or tissue; (c) determining the reads ratio or RPKM as compared to a control sample from a subject without the cell or tissue damage, wherein the reads ratio or RPKM having an above threshold range prior to a first treatment indicates that the subject has damage in the specific cell-type or tissue, and wherein the reads ratio or RPKM after a first time point after a first treatment having a below threshold range indicates the treatment is effective, and wherein the reads ratio or RPKM after a second time point after the first treatment having a higher threshold range indicates that the treatment is effective at the first time point but recurred at the second time point.

20. The method of claim 19 wherein the read-level or fragment-level cfDNA methylation sequencing reads are obtained by one or more of the following methods: (i) short-read WGBS for whole genome methylation profiling; (ii) short-read EM-seq for whole genome methylation profiling; (iii) Long-read sequencing based on PacBio SMRT for whole genome methylation profiling; (iv) Long-read sequencing based on Oxford Nanopore for whole genome methylation profiling; and (v) targeted sequencing based on panel of probes or PCR amplicon sequencing to capture specific to cell type or tissue-specific marker regions.

21. The method of claim 19 wherein the step of normalizing comprises: (i) relative cell type fraction across all marker regions; (ii) relative cell type fraction within corresponding marker regions; (iii) reads abundance normalized by sequencing depth across all marker regions and (iv) reads abundance normalized by sequencing depth at specific marker region.

22. The method of claim 19 wherein the disorder could be varieties of organ and tissue involved pathological conditions, including but not limited to graft rejections in organ transplantations, immune related diseases such as lupus nephritis and myocarditis, infection, cancer and radiation induced tissue damages of the subject.

23. The method of claim 19, wherein the cfDNA sample is obtained or derived from body fluids, including but not limited to a plasma sample, a blood sample, a saliva sample, an amniotic fluid sample, a cystic fluid sample, a spinal fluid sample, a brain fluid sample, a urine sample, a sweat sample, or a tears sample from the subject.

24. The method of claim 19, wherein the organ or tissue is adipose, bladder, colon, skin, stomach, heart, kidney, liver, lung, brain, pancreas, prostate, small intestine, thyroid or a combination thereof.

25. The method of claim 19, wherein the disorder is cancer and the tissue is liver cancer tissue, lung cancer tissue, kidney cancer tissue, colon cancer tissue, small intestines cancer tissue, pancreas cancer tissue, adrenal glands cancer tissue, esophagus cancer tissue, adipose cancer tissue, heart cancer tissue, brain cancer tissue, placenta cancer tissue, or combinations thereof.

26. The method of claim 19, wherein the treatment is chemotherapy, radiation therapy, immunotherapy, target therapy and tumor resection.
